(19) United States
(12) Patent Application Publication
(10) Pub. No.: US 2025/0198924 A1
(43) Pub. Date: Jun. 19, 2025

(54) INFORMATION PROCESSING DEVICE, BIOLOGICAL SAMPLE ANALYSIS SYSTEM, AND BIOLOGICAL SAMPLE ANALYSIS METHOD

(71) Applicant: SONY GROUP CORPORATION, TOKYO (JP)

(72) Inventors: NOE KANEKO, TOKYO (JP); KENJI IKEDA, TOKYO (JP); TETSURO KUWAYAMA, TOKYO (JP); KAZUHIRO NAKAGAWA, TOKYO (JP); TOMOHIKO NAKAMURA, TOKYO (JP)

(21) Appl. No.: 18/836,517

(22) PCT Filed: Feb. 9, 2023
(86) PCT No.: PCT/JP2023/004378

§ 371 Date: Feb. 28, 2025

(30) Foreign Application Priority Data
Feb. 16, 2022 (JP) 2022-022096

(51) Int. Cl. G01N 21/64; G01N 33/50; G01N 33/52

(52) U.S. Cl. CPC G01N 21/64; G01N 33/5008; G01N 33/52

(57) ABSTRACT

An information processing device according to an aspect of the present disclosure includes an acquisition unit that acquires a fluorescence spectrum derived from a biological sample and position information of the biological sample from a sample including the biological sample, an identification unit (133b) that identifies, from the fluorescence spectrum, information regarding a plurality of different biomarkers of the biological sample associated with the position information of the biological sample, and a correlation analysis unit (133d) that performs matrix decomposition processing corresponding to a combination of the plurality of biomarkers on the information regarding the plurality of biomarkers and outputs a correlation of the information regarding the plurality of biomarkers.

### FIELD

The present disclosure relates to an information processing device, a biological sample analysis system, and a biological sample analysis method.

### BACKGROUND

Currently, as indicated in the process of the cancer immunity cycle, it is considered that an interaction between cells (for example, contact between cells) triggers the progression or expulsion of cancer. However, it is difficult to algorithmize these relationships, and for example, it is difficult to obtain an algorithm that satisfies a biological aspect. Although it is important to perform correlation analysis between biomarkers so as to grasp the above-described interaction between cells, in a conventional clustering method of clustering a plurality of biomarkers (for example, hierarchical clustering, soft clustering, and the like), cell position information is not included, and information of multicolored biomarker staining is not fully utilized. For example, Non Patent Literature 1 proposes a method of measuring a distance between a certain cell and a phenotypic cell in a pathological diagnosis image, and measuring the number of cells and density.

### CITATION LIST

### Non Patent Literature

Non Patent Literature 1: SNEHA BERRY and 7 others, Analysis of multispectral imaging with the AstroPath platform informs efficacy of PD-1 blockade, (online), Science, 11 Jun. 2021, Vol 372, Issue 6547, (searched on Nov. 22, 2021), the Internet <URL: https://www.science.org/doi/10.1126/science.aba2609>

### SUMMARY

### Technical Problem

However, in Non Patent Literature 1, the relationship between a certain cell and a phenotypic cell is a one-to-one relationship and there is only one phenotypic cell type, and the method of Non Patent Literature 1 is not a method of obtaining a correlation between a plurality of cell types. For this reason, a method for obtaining a correlation among a plurality of cell types, that is, a correlation among a plurality of biomarkers is required.

Therefore, the present disclosure proposes an information processing device, a biological sample analysis system, and a biological sample analysis method capable of obtaining a correlation between a plurality of biomarkers.

### Solution to Problem

An information processing device according to an aspect of the present disclosure includes: an acquisition unit that acquires a fluorescence spectrum derived from a biological sample and position information of the biological sample from a sample including the biological sample; an identification unit that identifies, from the fluorescence spectrum, information regarding a plurality of different biomarkers of the biological sample associated with the position information of the biological sample; and a correlation analysis unit that performs matrix decomposition processing corresponding to a combination of the plurality of biomarkers on the information regarding the plurality of biomarkers and outputs a correlation of the information regarding the plurality of biomarkers.

A biological sample analysis system according to an aspect of the present disclosure includes: an imaging device that acquires a specimen image of a sample including a biological sample; and an information processing device that processes the specimen image, wherein the information processing device includes an acquisition unit that acquires a fluorescence spectrum derived from the biological sample and position information of the biological sample from the specimen image, an identification unit that identifies, from the fluorescence spectrum, information regarding a plurality of different biomarkers of the biological sample associated with the position information of the biological sample, and a correlation analysis unit that performs matrix decomposition processing corresponding to a combination of the plurality of biomarkers on the information regarding the plurality of biomarkers and outputs a correlation of the information regarding the plurality of biomarkers.

A biological sample analysis method according to an aspect of the present disclosure includes: acquiring a fluorescence spectrum derived from a biological sample and position information of the biological sample from a sample including the biological sample; identifying, from the fluorescence spectrum, information regarding a plurality of different biomarkers of the biological sample associated with the position information of the biological sample; and performing matrix decomposition processing corresponding to a combination of the plurality of biomarkers on the information regarding the plurality of biomarkers and outputting a correlation of the information regarding the plurality of biomarkers.

### BRIEF DESCRIPTION OF DRAWINGS

FIG. 1 is a diagram illustrating an example of a schematic configuration of an information processing system according to a first embodiment.

FIG. 2 is a flowchart illustrating an example of a flow of overall processing by an information processing device according to the first embodiment.

FIG. 3 is a diagram illustrating an example of a schematic configuration of a space analysis unit according to the first embodiment.

FIG. 4 is a flowchart illustrating an example of a flow of processing of correlation analysis of multi-biomarkers according to the first embodiment.

FIG. 5 is a diagram for describing Example 1 of samples according to the first embodiment.

FIG. 6 is a diagram illustrating an example of a positive cell rate of each block of AF488_CD7 according to the first embodiment.

FIG. 7 is a diagram illustrating an example of a positive cell rate for each block of AF555_CD3 according to the first embodiment.

FIG. 8 is a diagram illustrating an example of a positive cell rate for each block after sorting AF488_CD7 according to the first embodiment.

FIG. 9 is a diagram illustrating an example of a positive cell rate for each block after sorting AF647_CD5 according to the first embodiment.

FIG. 10 is a diagram for describing Example 1 of joint non-negative matrix factorization (JNMF) according to the first embodiment.

FIG. 11 is a diagram for describing a comparison between a classification result of W according to the first embodiment and an actual image.

FIG. 12 is a diagram for describing Example 2 of samples according to the first embodiment.

FIG. 13 is a diagram for describing Example 2 of the JNMF according to the first embodiment.

FIG. 14 is a diagram for describing Example 3 of the JNMF according to the first embodiment.

FIG. 15 is a diagram for describing Example 4 of the JNMF according to the first embodiment.

FIG. 16 is a diagram for describing Example 5 of the JNMF according to the first embodiment.

FIG. 17 is a diagram for describing determination of the number of clusters according to the first embodiment.

FIG. 18 is a diagram illustrating a result W of executing JNMF according to a second embodiment a plurality of times.

FIG. 19 is a diagram for describing an image of clustering according to the second embodiment.

FIG. 20 is a flowchart illustrating an example of a flow of processing of initial value calculation according to the second embodiment.

FIG. 21 is a diagram for describing types of cancer microenvironment (TME) according to the second embodiment.

FIG. 22 is a diagram for describing a simulation data set according to the second embodiment.

FIG. 23 is a diagram for describing a result of executing the JNMF on the samples 1 to 8 according to the second embodiment.

FIG. 24 is a diagram for describing that the JNMF is performed on a simulation data set obtained by adding a new sample 9 to the existing samples 1 to 8 according to the second embodiment.

FIG. 25 is a first diagram for describing an outline of initial values according to the second embodiment.

FIG. 26 is a second diagram for describing the outline of the initial values according to the second embodiment.

FIG. 27 is a diagram for describing a least squares method (LSM) according to the second embodiment.

FIG. 28 is a diagram for describing a first calculation method according to the second embodiment.

FIG. 29 is a diagram for describing a result of the JNMF using an initial value calculated by the first calculation method according to the second embodiment.

FIG. 30 is a diagram for describing a second calculation method according to the second embodiment.

FIG. 31 is a diagram for describing a result of the JNMF using an initial value calculated by the second calculation method according to the second embodiment.

FIG. 32 is a diagram for describing a third calculation method according to the second embodiment.

FIG. 33 is a diagram for describing a result of the JNMF using an initial value calculated by a third calculation method according to the second embodiment.

FIG. 34 is a diagram for describing a fourth calculation method according to the second embodiment.

FIG. 35 is a diagram for describing a result of the JNMF using an initial value calculated by a fourth calculation method according to the second embodiment.

FIG. 36 is a diagram for describing a fifth calculation method according to the second embodiment.

FIG. 37 is a diagram for describing a result of the JNMF using an initial value calculated by a fifth calculation method according to the second embodiment.

FIG. 38 is a diagram illustrating an example of a schematic configuration of a fluorescence observation device.

FIG. 39 is a diagram illustrating an example of a schematic configuration of an observation unit.

FIG. 40 is a diagram illustrating an example of a sample.

FIG. 41 is an enlarged diagram illustrating a region where a sample is irradiated with line illumination.

FIG. 42 is a diagram schematically illustrating the overall configuration of a microscope system.

FIG. 43 is a diagram illustrating an example of an imaging method.

FIG. 44 is a diagram illustrating an example of an imaging method.

FIG. 45 is a diagram illustrating an example of a schematic configuration of hardware of the information processing device.

### DESCRIPTION OF EMBODIMENTS

Hereinafter, an embodiment of the present disclosure (including examples and modifications) will be described in detail with reference to the drawings. Note that the device, the system, the method, and the like according to the present disclosure are not limited by the embodiment. Further, in the present description and the drawings, components having substantially the same functional configuration are basically denoted by the same reference numerals, and redundant description is omitted.

One or more embodiments (examples and modifications) described below can each be implemented independently. On the other hand, at least some of the plurality of embodiments described below may be appropriately combined with at least some of other embodiments. The plurality of embodiments may include novel features different from each other. Therefore, the plurality of embodiments can contribute to solving different objects or problems, and can exhibit different effects.

The present disclosure will be described according to the following order of items.

### 1. First Embodiment

1-1. Configuration example of information processing system
1-2. Processing example of information processing device
1-3. Processing example of correlation analysis of multi-biomarker
1-4. Specific examples of correlation analysis of multi-biomarkers
1-5. Operation and effect
2. Second Embodiment
2-1. Summary of second embodiment
2-2. Processing example of initial value calculation
2-3. Example of initial value calculation
2-3-1. Simulation data set
2-3-2. Calculation method of initial value
2-4. Operation and effect
3. Other embodiments
4. Application example
5. Application example
6. Configuration example of hardware
7. Appendix

### 1. First Embodiment

### <1-1. Configuration Example of Information Processing System>

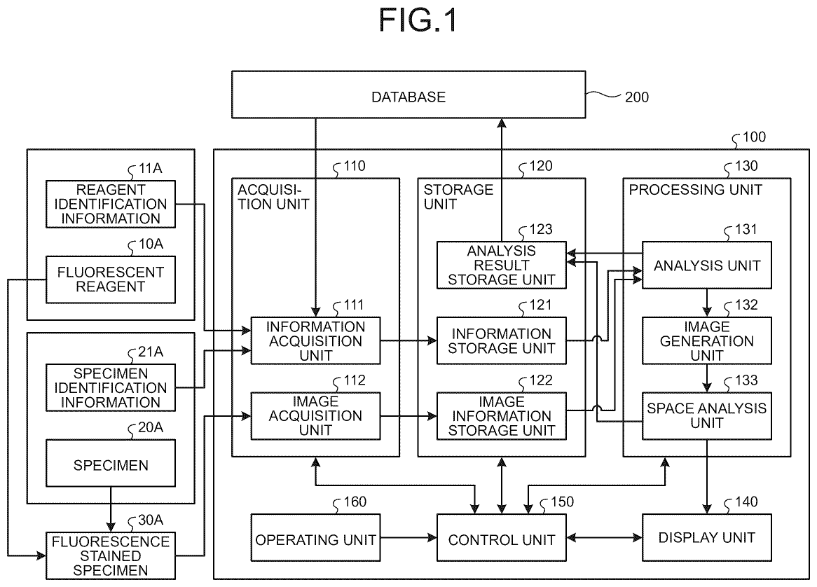

A configuration example of an information processing system according to the present embodiment will be described with reference to FIG. 1. FIG. 1 is a diagram illustrating an example of a schematic configuration of an information processing system according to the present embodiment. The information processing system is an example of a biological sample analysis system.

As illustrated in FIG. 1, the information processing system according to the present embodiment includes an information processing device 100 and a database 200. As inputs to the information processing system, there are a fluorescent reagent 10A, a specimen 20A, and a fluorescence stained specimen 30A.

### (Fluorescent Reagent 10A)

The fluorescent reagent 10A is a chemical used for staining the specimen 20A. The fluorescent reagent 10A is, for example, a fluorescent antibody (primary antibodies used for direct labeling or secondary antibodies used for indirect labeling), a fluorescent probe, a nuclear staining reagent, or the like, but the type of the fluorescent reagent 10A is not particularly limited thereto. Further, the fluorescent reagent 10A is managed with identification information (hereinafter referred to as “reagent identification information 11A”) that can identify the fluorescent reagent 10A (and the production lot of the fluorescent reagent 10A). The reagent identification information 11A is, for example, bar code information (one-dimensional bar code information, two-dimensional bar code information, or the like), but is not limited thereto. The properties of the fluorescent reagent 10A are different for each production lot depending on the production method, the state of cells from which the antibody is acquired, and the like even for the same (same type of) products. For example, in the fluorescent reagent 10A, spectrum information, quantum yield, or fluorescent labeling rate (also referred to as “F/P value: Fluorescein/Protein” and refers to the number of fluorescent molecules that label an antibody) or the like is different for each production lot. Accordingly, in the information processing system according to the present embodiment, the fluorescent reagent 10A is managed for each production lot by being attached with the reagent identification information 11A (in other words, reagent information of each fluorescent reagent 10A is managed for each production lot). Thus, the information processing device 100 can separate a fluorescence signal and an autofluorescence signal in consideration of a slight difference in property appearing for each production lot. Note that the management of the fluorescent reagent 10A in units of production lots is merely an example, and the fluorescent reagent 10A may be managed in units finer than the production lots.

### (Specimen 20A)

The specimen 20A is prepared for the purpose of pathological diagnosis, clinical examination, or the like from a specimen or a tissue sample collected from a human body. For the specimen 20A, the type of the tissue being used (for example, an organ or a cell), the type of disease of interest, the attributes of the subject (for example, age, sex, blood type, or race), or the subject's daily habits (for example, an eating habit, an exercise habit, or a smoking habit) are not particularly limited. Further, the specimen 20A is managed with identification information (hereinafter referred to as “specimen identification information 21A”) that can identify each specimen 20A. As is the reagent identification information 11A, the specimen identification information 21A is, for example, bar code information (one-dimensional bar code information, two-dimensional bar code information, or the like), but is not limited thereto. The properties of the specimen 20A vary depending on the type of the tissue being used, the type of the target disease, the attributes of the subject, the daily habits of the subject, or the like. For example, in the specimen 20A, a measurement channel, spectrum information, and the like varies depending on the type of the tissue being used, and the like. Accordingly, in the information processing system according to the present embodiment, the specimen 20A is individually managed by being attached with the specimen identification information 21A. Thus, the information processing device 100 can separate the fluorescence signal and the autofluorescence signal in consideration of a slight difference in property appearing for each specimen 20A.

### (Fluorescence Stained Specimen 30A)

The fluorescence stained specimen 30A is prepared by staining the specimen 20A with the fluorescent reagent 10A. In the present embodiment, it is assumed that, in the fluorescence stained specimen 30A, the specimen 20A is stained with at least one or more fluorescent reagents 10A, but the number of fluorescent reagents 10A used for staining is not particularly limited. Further, the staining method is determined by a combination of each of the specimen 20A and the fluorescent reagent 10A, and the like, and is not particularly limited. The fluorescence stained specimen 30A is input to the information processing device 100 and imaged.

### (Information Processing Device 100)

As illustrated in FIG. 1, the information processing device 100 includes an acquisition unit 110, a storage unit 120, a processing unit 130, a display unit 140, a control unit 150, and an operating unit 160.

### (Acquisition Unit 110)

The acquisition unit 110 is configured to acquire information used for various processes of the information processing device 100. As illustrated in FIG. 1, the acquisition unit 110 includes an information acquisition unit 111 and an image acquisition unit 112.

### (Information Acquisition Unit 111)

The information acquisition unit 111 is configured to acquire various types of information such as reagent information and specimen information. More specifically, the information acquisition unit 111 acquires the reagent identification information 11A attached to the fluorescent reagent 10A used for generating the fluorescence stained specimen 30A and the specimen identification information 21A attached to the specimen 20A. For example, the information acquisition unit 111 acquires the reagent identification information 11A and the specimen identification information 21A using a barcode reader or the like. Then, the information acquisition unit 111 acquires the reagent information on the basis of the reagent identification information 11A and the specimen information on the basis of the specimen identification information 21A from the database 200. The information acquisition unit 111 stores the acquired information in an information storage unit 121 described later.

### (Image Acquisition Unit 112)

The image acquisition unit 112 is configured to acquire image information of the fluorescence stained specimen 30A (the specimen 20A stained with at least one fluorescent reagent 10A). More specifically, the image acquisition unit 112 includes any imaging element (for example, a CCD, a CMOS, or the like), and acquires the image information by imaging the fluorescence stained specimen 30A using the imaging element. Here, it should be noted that the “image information” is a concept including not only the image of the fluorescence stained specimen 30A itself but also a measurement value that is not visualized as an image. For example, the image information may include information regarding a wavelength spectrum (hereinafter referred to as fluorescence spectrum) of the fluorescence emitted from the fluorescence stained specimen 30A. The image acquisition unit 112 stores the image information in an image information storage unit 122 described later.

### (Storage Unit 120)

The storage unit 120 is configured to store (save) information used for various processes of the information processing device 100 or information output by the various processes. As illustrated in FIG. 1, the storage unit 120 includes an information storage unit 121, an image information storage unit 122, and an analysis result storage unit 123.

### (Information Storage Unit 121)

The information storage unit 121 is configured to store various types of information such as reagent information and specimen information acquired by the information acquisition unit 111. Note that, after an analysis process by an analysis unit 131 and a generation process of the image information by an image generation unit 132 (a reconstruction process of the image information) which will be described later is finished, the information storage unit 121 may increase the free space by deleting the reagent information and the specimen information used for the process.

### (Image Information Storage Unit 122)

The image information storage unit 122 is configured to store the image information of the fluorescence stained specimen 30A acquired by the image acquisition unit 112. Note that, after the analysis process by the analysis unit 131 and the generation process of the image information by the image generation unit 132 (the reconstruction process of the image information) is finished, as does the information storage unit 121, the image information storage unit 122 may increase the free space by deleting the image information used for the process.

### (Analysis Result Storage Unit 123)

The analysis result storage unit 123 is configured to store a result of the analysis process performed by the analysis unit 131 or the space analysis unit 133 described later. For example, the analysis result storage unit 123 stores the fluorescence signal of the fluorescent reagent 10A or the autofluorescence signal of the specimen 20A separated by the analysis unit 131, a correlation analysis result or an effect prediction result (effect estimation result) obtained by the space analysis unit 133, and the like. In addition, the analysis result storage unit 123 separately provides the result of the analysis process to the database 200 in order to improve analysis accuracy by machine learning or the like. Note that, after providing the result of the analysis process to the database 200, the analysis result storage unit 123 may increase the free space by appropriately deleting the result of the analysis process stored therein.

### (Processing Unit 130)

The processing unit 130 is a functional configuration that performs various processes using the image information, the reagent information, and the specimen information. As illustrated in FIG. 1, the processing unit 130 includes an analysis unit 131, an image generation unit 132, and a space analysis unit 133.

### (Analysis Unit 131)

The analysis unit 131 is configured to perform various analysis processes using the image information, the specimen information, and the reagent information. For example, the analysis unit 131 performs processing (color separation processing) of separating the autofluorescence signal of the specimen 20A and the fluorescence signal of the fluorescent reagent 10A from the image information on the basis of the specimen information and the reagent information.

Specifically, the analysis unit 131 recognizes one or more elements constituting the autofluorescence signal on the basis of the measurement channel included in the specimen information. For example, the analysis unit 131 recognizes one or more autofluorescence components constituting the autofluorescence signal. Then, the analysis unit 131 predicts the autofluorescence signal included in the image information using the spectrum information of these autofluorescence components included in the specimen information. Then, the analysis unit 131 separates the autofluorescence signal and the fluorescence signal from the image information on the basis of the spectrum information of the fluorescence component of the fluorescent reagent 10A included in the reagent information and the predicted autofluorescence signal.

Here, when the specimen 20A is stained with two or more fluorescent reagents 10A, the analysis unit 131 separates the fluorescence signal of each of these two or more fluorescent reagents 10A from the image information (or the fluorescence signal after being separated from the autofluorescence signal) on the basis of the specimen information and the reagent information. For example, the analysis unit 131 separates the fluorescence signal of each of the fluorescent reagents 10A from the entire fluorescence signal after being separated from the autofluorescence signal by using the spectrum information of the fluorescence component of each of the fluorescent reagents 10A included in the reagent information.

In addition, in a case where the autofluorescence signal is constituted by two or more autofluorescence components, the analysis unit 131 separates the autofluorescence signal of each autofluorescence component from the image information (or the autofluorescence signal after being separated from the fluorescence signal) on the basis of the specimen information and the reagent information. For example, the analysis unit 131 separates the autofluorescence signal of each autofluorescence component from the entire autofluorescence signal after being separated from the fluorescence signal by using the spectrum information of each autofluorescence component included in the specimen information.

The analysis unit 131 that has separated the fluorescence signal and the autofluorescence signal performs various processes using these signals. For example, the analysis unit 131 may extract the fluorescence signal from the image information of the other specimen 20A by performing a subtraction process (also referred to as a “background subtraction process”) on the image information of the other specimen 20A using the autofluorescence signal after separation. In a case where there is a plurality of specimens 20A that is the same or similar in terms of the tissue being used for the specimen 20A, the type of the target disease, the attributes of the subject, the daily habit of the subject, and the like, there is a high possibility that the autofluorescence signals of these specimens 20A are similar. The similar specimen 20A mentioned here includes, for example, a tissue section before staining of a tissue section to be stained (hereinafter referred to as a section), a section adjacent to a stained section, a section different from a stained section in the same block (sampled from the same place as the stained section), or a section in a different block in the same tissue (sampled from a different place from the stained section) or the like), a section sampled from a different patient, or the like. Accordingly, when the autofluorescence signal can be extracted from a certain specimen 20A, the analysis unit 131 may extract the fluorescence signal from the image information of the other specimen 20A by removing the autofluorescence signal from the image information of the other specimen 20A. Furthermore, when calculating the S/N value using the image information of the other specimen 20A, the analysis unit 131 can improve the S/N value by using the background after removing the autofluorescence signal.

In addition to the background subtraction process, the analysis unit 131 can perform various processes using the fluorescence signal or autofluorescence signal after separation. For example, the analysis unit 131 can analyze the fixation state of the specimen 20A using these signals, and can perform segmentation (or region division) for recognizing a region of an object (for example, a cell, intracellular structure (cytoplasm, cell membrane, nucleus, or the like), or tissue (tumor site, non-tumor site, connective tissue, blood vessel, blood vessel wall, lymphatic vessel, fibrosed structure, necrosis, and the like)) included in the image information.

### (Image Generation Unit 132)

The image generation unit 132 is configured to generate image information on the basis of the analysis result obtained by the analysis unit 131. In addition, the image generation unit 132 generates (reconstructs) image information on the basis of the fluorescence signal or the autofluorescence signal separated by the analysis unit 131. For example, the image generation unit 132 can generate image information including only a fluorescence signal or image information including only an autofluorescence signal. At that time, in a case where the fluorescence signal is constituted by a plurality of fluorescence components or the autofluorescence signal is constituted by a plurality of autofluorescence components, the image generation unit 132 can generate the image information in units of respective components. Furthermore, in a case where the analysis unit 131 performs various processes (for example, analysis of the fixation state of the specimen 20A, segmentation, calculation of the S/N value, or the like) using the fluorescence signal or the autofluorescence signal after separation, the image generation unit 132 may generate the image information indicating a result of the process. With this configuration, distribution information of the fluorescent reagent 10A labeled with a target molecule or the like, that is, a two-dimensional spread and intensity of fluorescence, a wavelength, and a positional relationship thereof are visualized, and in particular, in a tissue image analysis region in which information of a target substance is complicated, the visibility of a doctor or a researcher who is the user can be improved.

In addition, the image generation unit 132 may perform control to distinguish the fluorescence signal with respect to the autofluorescence signal on the basis of the fluorescence signal or the autofluorescence signal separated by the analysis unit 131, and generate the image information. Specifically, the image information may be generated by performing control of improving the luminance of the fluorescence spectrum of the fluorescent reagent 10A labeled with the target molecule or the like, extracting and changing the color of only the fluorescence spectrum of the labeled fluorescent reagent 10A, extracting the fluorescence spectrum of two or more fluorescent reagents 10A from the specimen 20A labeled with two or more fluorescent reagents 10A and changing the color of each of them to another color, extracting and dividing or subtracting only the autofluorescence spectrum of the specimen 20A, improving the dynamic range, and the like. Thus, the user can clearly distinguish color information derived from the fluorescent reagent bound to the target substance, and the visibility of the user can be improved.

### (Space Analysis Unit 133)

The space analysis unit 133 performs processing of analyzing a correlation between a plurality of biomarkers (for example, between tissues) from image information after color separation, and processing of predicting a drug effect on the basis of a correlation analysis result that is an analysis result of the correlation. For example, the space analysis unit 133 performs clustering analysis on a specimen image stained with a plurality of biomarkers while maintaining space information, that is, position information, thereby analyzing the correlation between the biomarkers. Such a correlation analysis process and a drug effect prediction process of multi-biomarkers will be described in detail later. Note that the space analysis unit 133 can generate image information including, for example, a correlation analysis result, an effect prediction result (effect estimation result), and the like obtained by processing. The space analysis unit 133 also functions as an image generation unit.

### (Display Unit 140)

The display unit 140 is configured to present image information generated by the image generation unit 132, the space analysis unit 133, or the like to the user by displaying the image information on the display. Note that the type of display used as the display unit 140 is not particularly limited. Furthermore, although not described in detail in the present embodiment, the image information generated by the image generation unit 132, the space analysis unit 133, or the like may be presented to the user by being projected by a projector or printed by a printer (in other words, a method of outputting the image information is not particularly limited).

### (Control Unit 150)

The control unit 150 is a functional configuration that comprehensively controls overall processing performed by the information processing device 100. For example, the control unit 150 controls the start, end, and the like of various processes (for example, imaging process, analysis process, generation process of image information (reconstruction process of image information), display process of image information, and the like of the fluorescence stained specimen 30A) as described above on the basis of an operation input by the user performed via the operating unit 160. Note that the control content of the control unit 150 is not particularly limited. For example, the control unit 150 may control processing generally performed in a general-purpose computer, a PC, a tablet PC, or the like (for example, processing related to an operating system (OS)).

### (Operating Unit 160)

The operating unit 160 is configured to receive an operation input from a user. More specifically, the operating unit 160 includes various input units such as a keyboard, a mouse, a button, a touch panel, or a microphone, and the user can perform various inputs to the information processing device 100 by operating these input units. Information regarding the operation input performed via the operating unit 160 is provided to the control unit 150.

### (Database 200)

The database 200 is a device that manages the specimen information, the reagent information, and results of the analysis process. More specifically, the database 200 manages the specimen identification information 21A and the specimen information and the reagent identification information 11A and the reagent information in association with each other. Thus, the information acquisition unit 111 can acquire the specimen information on the basis of the specimen identification information 21A of the specimen 20A to be measured and the reagent information from the database 200 on the basis of the reagent identification information 11A of the fluorescent reagent 10A.

As described above, the specimen information managed by the database 200 is information including the measurement channel and the spectrum information unique to the autofluorescence component included in the specimen 20A. However, in addition to these, the specimen information may include target information for each specimen 20A, specifically, information regarding the type of the tissue being used (for example, an organ, a cell, blood, a body fluid, ascites, or pleural effusion), the type of disease to be a target, attributes of the subject (for example, age, sex, blood type, or race), or the subject's daily habits (for example, an eating habit, an exercise habit, or a smoking habit), and the information including the measurement channel and the spectrum information unique to the autofluorescence component included in the specimen 20A and the target information may be associated with each specimen 20A. Thus, the information including the measurement channel and the spectrum information unique to the autofluorescence component included in the specimen 20A can be easily traced from the target information, and for example, the analysis unit 131 can be caused to execute a similar separation process performed in the past from the similarity of the target information in the plurality of specimens 20A, so that the measurement time can be shortened. Note that, the “tissue being used” is not particularly limited to a tissue collected from the subject, and may include an in vivo tissue or a cell line of a human, an animal, or the like, and a solution, a solvent, a solute, and a material contained in an object to be measured.

Further, the reagent information managed by the database 200 is information including the spectrum information of the fluorescent reagent 10A as described above, but in addition to this, the reagent information may include information regarding the fluorescent reagent 10A, such as a production lot, a fluorescence component, an antibody, a clone, a fluorescent labeling rate, a quantum yield, a fading coefficient (information indicating easiness of reducing the fluorescence intensity of the fluorescent reagent 10A), and an absorption cross-sectional area (or a molar absorption coefficient). Furthermore, the specimen information and the reagent information managed by the database 200 may be managed in different configurations, and in particular, the information regarding the reagent may be a reagent database that presents an optimal combination of reagents to the user.

Here, it is assumed that the specimen information and the reagent information are provided from a producer (manufacturer) or the like, or are independently measured in the information processing system according to the present disclosure. For example, the manufacturer of the fluorescent reagent 10A often does not measure and provide spectrum information, a fluorescent labeling rate, and the like for each production lot. Therefore, by uniquely measuring and managing these pieces of information in the information processing system according to the present disclosure, the separation accuracy of the fluorescence signal and the autofluorescence signal can be improved. In addition, in order to simplify the management, the database 200 may use a catalog value disclosed by a producer (manufacturer) or the like, a document value described in various documents, or the like as the specimen information and the reagent information (particularly the reagent information). However, in general, since the actual specimen information and reagent information are often different from the catalog value and the document value, it is more preferable that the specimen information and the reagent information are uniquely measured and managed in the information processing system according to the present disclosure as described above.

In addition, accuracy of the analysis process (for example, a separation process of the fluorescence signal and the autofluorescence signal, a correlation analysis process of multi-biomarkers, a prediction process of drug effect, and the like) can be improved by a machine learning technique or the like using the specimen information, the reagent information, and the results of the analysis process managed in the database 200. A subject that performs learning using a machine learning technique or the like is not particularly limited. For example, the analysis unit 131 generates a classifier or an estimator machine-learned by learning data using a neural network. Then, in a case where corresponding various types of information are newly acquired, the analysis unit 131 inputs these pieces of information to the classifier or the estimator to perform the separation process of the fluorescence signal and the autofluorescence signal, the correlation analysis process of the multi-biomarkers, and the prediction process of drug effect.

In addition, a method of obtaining similar processes performed in the past with higher accuracy than the predicted result, statistically or regressively analyzing the contents of processing (information, parameters, and the like used for the processing) in those processes, and improving the separation process of the fluorescence signal and the autofluorescence signal, the correlation analysis process of the multi-biomarkers, and the prediction process of the drug effect on the basis of the analysis result may be output. Note that the machine learning method is not limited to the above, and a known machine learning technique can be used. In addition, the separation process of the fluorescence signal and the autofluorescence signal, the correlation analysis process of the multi-biomarkers, and prediction process of drug effect may be performed by artificial intelligence. In addition, other various types of processes (for example, analysis of the fixation state of the specimen 20A, segmentation, or the like) may be improved by a machine learning technique or the like.

The configuration example of the information processing system according to the present embodiment has been described above. Note that the above-described configuration described with reference to FIG. 1 is merely an example, and the configuration of the information processing system according to the present embodiment is not limited to such an example. For example, the information processing device 100 may not necessarily include all of the functional configurations illustrated in FIG. 1. In addition, the information processing device 100 may include the database 200 therein. The functional configuration of the information processing device 100 can be flexibly modified according to specifications and operations.

In addition, the information processing device 100 may perform processing other than the processing described above. For example, when the reagent information includes information such as the quantum yield, the fluorescent labeling rate, and the absorption cross-sectional area (or the molar absorption coefficient) related to the fluorescent reagent 10A, the information processing device 100 may calculate the number of fluorescent molecules, the number of antibodies bound to fluorescent molecules, or the like in the image information by using the image information from which the autofluorescence signal has been removed and the reagent information.

### <1-2. Processing Example of Information Processing Device>

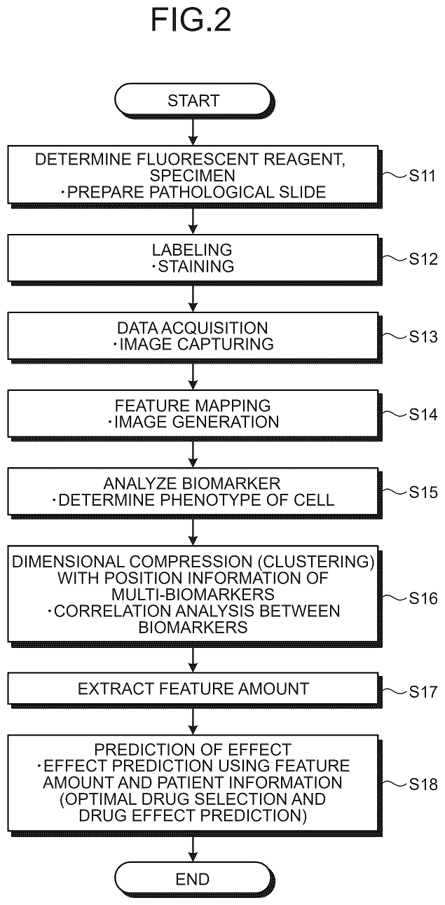

A processing example (entire flow) of the information processing device 100 according to the present embodiment will be described with reference to FIG. 2. FIG. 2 is a flowchart illustrating an example of an overall processing flow of the information processing device 100 according to the present embodiment.

As illustrated in FIG. 2, in step S11, the user determines the fluorescent reagent 10A and the specimen 20A to be used for analysis. For example, the user prepares a pathological slide (slice). In step S12, the user stains the specimen 20A using the fluorescent reagent 10A to prepare the fluorescence stained specimen 30A.

In step S13, the image acquisition unit 112 of the information processing device 100 images the fluorescence stained specimen 30A to acquire image information. In step S14, the analysis unit 131 separates the autofluorescence signal of the specimen 20A and the fluorescence signal of the Fluorescent reagent 10A from the image information on the basis of the specimen information and the reagent information, and the image generation unit 132 generates image information using the separated fluorescence signal. For example, the image generation unit 132 generates image information in which the autofluorescence signal is removed from the image information, or generates image information indicating a fluorescence signal for each fluorescent pigment. Note that the information acquisition unit 111 acquires the reagent information and the specimen information from the database 200 on the basis of the reagent identification information 11A attached to the fluorescent reagent 10A used for generating the fluorescence stained specimen 30A and the specimen identification information 21A attached to the specimen 20A.

In step S15, the space analysis unit 133 analyzes the biomarker from the image information after the color separation. For example, the space analysis unit 133 determines the phenotype of the cell. In step S16, the space analysis unit 133 performs dimensional compression (clustering) with position information of multi-biomarkers.

For example, the space analysis unit 133 performs dimensional compression with position information of multi-biomarkers and executes correlation analysis between biomarkers. In step S17, the space analysis unit 133 extracts a feature amount from the correlation between the biomarkers. In step S18, the space analysis unit 133 executes effect prediction of a drug (medicine) using the feature amount and patient information. For example, the space analysis unit 133 performs optimal drug selection, drug effect prediction, and the like using the feature amount and the patient information. The patient information may include, for example, patient identification information and information such as administration drug candidates for the patient. Processing in such steps S15 to S18 and a configuration of the space analysis unit 133 related to the processing will be described in detail later.

Note that the space analysis unit 133 may generate image information including an analysis result obtained in step S16, image information including the feature amount obtained in step S17, image information including the optimal drug selection and the drug effect prediction obtained in step S18, and the like. These pieces of image information are displayed by the display unit 140. Thus, the user can view and grasp various types of information displayed by the display unit 140.

Furthermore, each step in the flowchart illustrated in FIG. 2 is not necessarily processed in time series in the described order. That is, each step in the flowchart may be processed in an order different from the described order or may be processed in parallel. In addition, the information processing device 100 may also execute processing not illustrated in FIG. 2.

### <1-3. Processing Example of Correlation Analysis of Multi-Biomarkers>

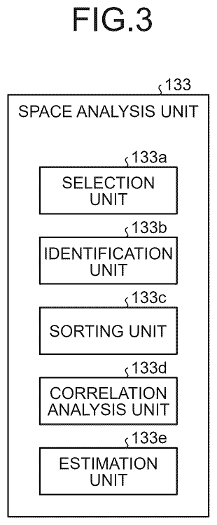

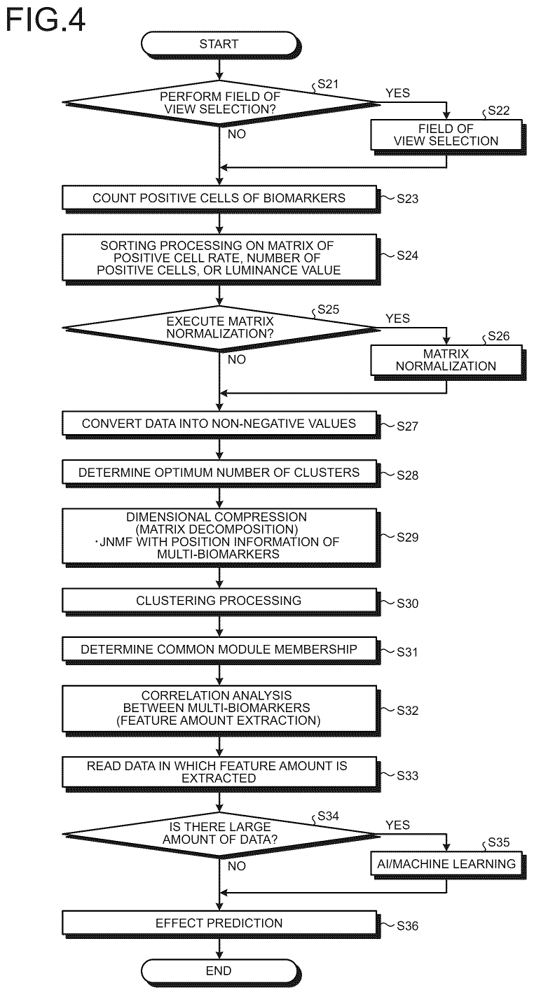

A processing example of correlation analysis of multi-biomarkers according to the present embodiment will be described with reference to FIGS. 3 and 4. FIG. 3 is a diagram illustrating an example of a schematic configuration of the space analysis unit 133 according to the present embodiment. FIG. 4 is a flowchart illustrating an example of a flow of processing of correlation analysis of multi-biomarkers according to the present embodiment.

As illustrated in FIG. 3, the space analysis unit 133 includes a selection unit 133a, an identification unit 133b, a sorting unit 133c, a correlation analysis unit 133d, and an estimation unit 133e.

The selection unit 133a determines a predetermined region (for example, a region of interest) of a sample (for example, a specimen image). The identification unit 133b extracts and identifies information (for example, the amount of positive cells) regarding a plurality of different biomarkers of a biological sample associated with position information of the biological sample in the predetermined region from a fluorescence spectrum derived from the biological sample in the predetermined region (for example, a predetermined field of view). The sorting unit 133c changes, on the basis of an arrangement order of a plurality of pieces of unit information (for example, blocks) included in the information regarding one biomarker out of the information regarding the plurality of biomarkers, an arrangement order of a plurality of pieces of unit information (for example, blocks) included in information regarding the other biomarkers. The correlation analysis unit 133d performs clustering processing on the information regarding the plurality of biomarkers in which the arrangement order of the unit information is changed, and outputs a correlation of the information regarding the plurality of biomarkers. The estimation unit 133e estimates efficacy to a patient of the administration drug candidate from the correlation of the information regarding the plurality of biomarkers and the administration drug candidate for the patient.

Here, the acquisition unit 110 (see FIG. 1) acquires a fluorescence spectrum derived from a biological sample and position information of the biological sample from a sample including the biological sample. The storage unit 120 stores the fluorescence spectrum derived from the biological sample and the position information of the biological sample. The fluorescence spectrum derived from the biological sample and the position information of the biological sample are used by the selection unit 133a. In addition, the acquisition unit 110 (see FIG. 1), that is, the information acquisition unit 111 acquires an administration drug candidate for a patient related to a biological sample. The storage unit 120 stores the administration drug candidate for the patient related to the biological sample. Information on the administration drug candidate for the patient related to the biological sample is used by the estimation unit 133e.

As illustrated in FIG. 4, in step S21, it is determined whether or not the selection unit 133a executes field of view selection (determination of a predetermined region) on the specimen image after the color separation. In step S22, the selection unit 133a executes field of view selection. In step S23, the identification unit 133b counts positive cells of biomarkers in the specimen image after the color separation or the selected field of view of the specimen image. For example, the identification unit 133b divides the specimen image after the color separation or the selected field of view of the specimen image into a matrix of block regions, and obtains a positive cell rate, the number of positive cells, or a luminance value for each block region. Thus, a matrix of the positive cell rate, the number of positive cells, or the luminance value is obtained. The matrix information also includes position information. Note that the positive cell rate is the number of positive cells with respect to the number of cells present per unit area. The number of positive cells is synonymous with the number of cells per unit area, that is, positive cell density.

In step S24, the sorting unit 133c performs sorting processing on the matrix of the positive cell rate, the number of positive cells, or the luminance value of another biomarker on the basis of the positive cell rate, the number of positive cells, or the luminance value of a certain biomarker. In step S25, the correlation analysis unit 133d determines whether or not to execute matrix normalization. In step S26, the correlation analysis unit 133d executes matrix normalization. In step S27, the correlation analysis unit 133d converts the data of the matrix into non-negative values. In step S28, the correlation analysis unit 133d determines the optimum number of clusters. For example, the optimum number of clusters may be automatically determined by the correlation analysis unit 133d, or may be set according to an input operation of the user to the operating unit 160.

In step S29, the correlation analysis unit 133d performs matrix decomposition processing on the data of the matrix. For example, the correlation analysis unit 133d executes dimensional compression (simultaneous decomposition of multiple matrices) with position information of multi-biomarkers by joint non-negative matrix factorization (JNMF (jNMF)). In step S30, the correlation analysis unit 133d executes clustering from the result of dimensional compression. In step S31, the correlation analysis unit 133d determines a membership of a common module. In step S32, the correlation analysis unit 133d performs correlation analysis between multi-biomarkers. For example, the correlation analysis unit 133d extracts a feature amount. In step S33, the estimation unit 133e reads data from which a feature amount has been extracted. In step S34, the estimation unit 133e determines whether or not there is a large amount of data. In step S35, the estimation unit 133e executes AI/machine learning. In step S36, the estimation unit 133e executes effect prediction.

Here, in step S26, in a case where the magnitude of a value is greatly different between samples or between biomarkers, the size of the matrix is normalized in such a manner that the sum of squares of each matrix is the same. Furthermore, in step S35, the estimation unit 133e can read the extracted feature amount and determine the phenotype of the cell. The estimation unit 133e assumes a phenotype of cancer of a patient together with patient information, and executes optimal drug (medicine) selection and drug effect prediction, or uses the phenotype for patient selection such as a clinical trial. The estimation unit 133e functions as a predictor by AI/machine learning. Note that, when the effect prediction is performed, prediction by AI or the like may be executed from the extracted feature amount.

Note that each step in the flowchart of FIG. 4 is not necessarily processed in time series in the described order. That is, each step in the flowchart may be processed in an order different from the described order or may be processed in parallel. In addition, the information processing device 100 may also execute processing not illustrated in FIG. 4.

### <1-4. Specific Examples of Correlation Analysis of Multi-Biomarker>

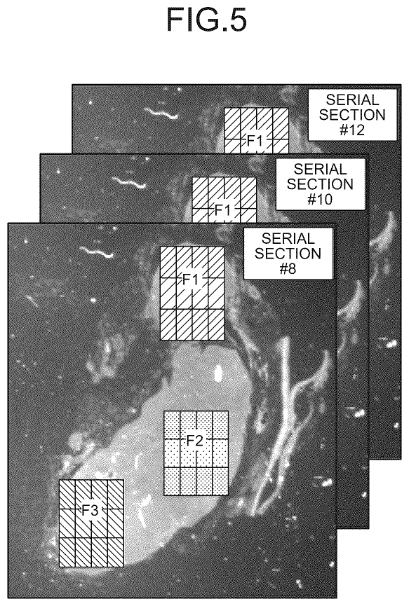

A specific example of correlation analysis of multi-biomarkers with respect to a specimen image according to the present embodiment will be described with reference to FIGS. 5 to 17.

FIG. 5 is a diagram for describing Example 1 of the sample according to the present embodiment. As illustrated in FIG. 5, three serial sections (section Nos. #8, #10, and #12) are used. These serial sections (specimen images) are samples of tonsils. Specifically, samples of tonsils stained with AF488_CD7, AF555_CD3, AF647_CD5, and DAPI (4′,6-Diamidino-2-phenylindole, dihydrochloride) are used, and three serial sections of the samples are used.

### (Field of View Selection Processing)

The selection unit 133a divides three fields of view (F1, F2, and F3) different for each of serial sections (section Nos. #8, #10, and #12) into regions of 3 bands×4 blocks (total: 12 blocks, 1 block 610×610 pixels), and uses a total of 108 blocks as data. This region is a predetermined region (region of interest), and the predetermined region is set in advance. The predetermined region may be settable by an input operation of the user on the operating unit 160. Note that the position information of each region in one section is two-dimensional information (position information in a plane), and the position information of each region in serial sections is three-dimensional information (space information). For example, the position information includes XY coordinates, Z coordinates, and the like based on the pixel.

### (Positive Cell Amount Calculation Processing)

The identification unit 133b obtains the positive cell rate of each biomarker for each region (block). For example, the identification unit 133b obtains the positive cell rate (%) of each biomarker for each region. Thus, for example, respective positive cell rates of AF488_CD7, AF555_CD3, and AF647_CD5 are obtained. Note that the identification unit 133b may obtain a numerical value other than the positive cell rate, and for example, may obtain an average luminance value, the number of positive cells, or the like in the region.

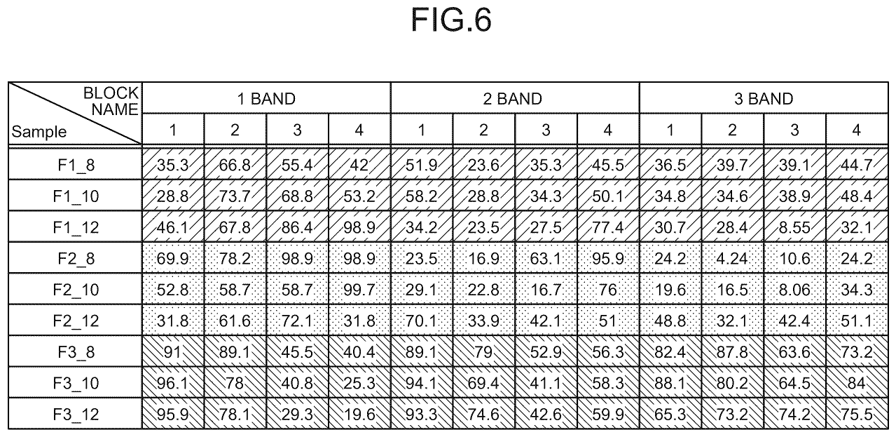

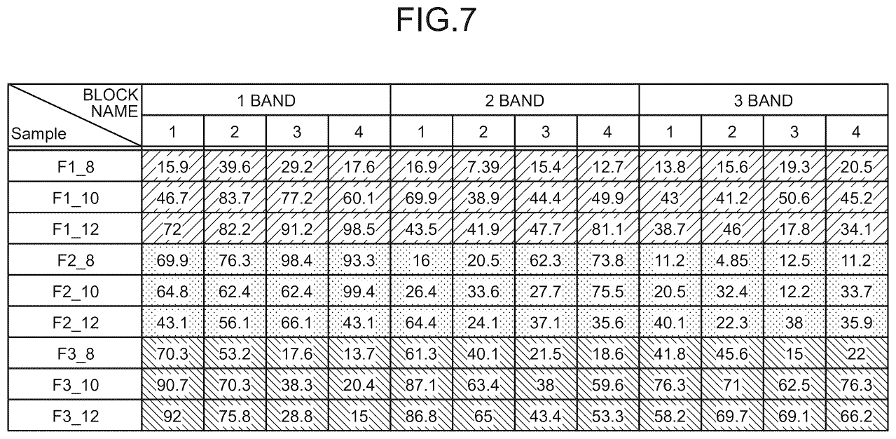

FIG. 6 is a diagram illustrating an example of the positive cell rate of each block of AF488_CD7 according to the present embodiment. FIG. 7 is a diagram illustrating an example of the positive cell rate of each block of AF555_CD3 according to the present embodiment. In these examples of FIGS. 6 and 7, the sample name is indicated by “field of view serial section number” (the same applies to the following drawings), and the filling pattern is changed for each field of view (F1, F2, or F3) for easy understanding. This filling pattern corresponds to the filling pattern of FIG. 5.

### (Sorting Process)

The sorting unit 133c sorts blocks (spaces) of other biomarkers for each sample on the basis of the positive cell rate of a specific biomarker. For example, the sorting unit 133c sorts blocks of other biomarkers in the row direction for each sample on the basis of the positive cell rate of a specific biomarker. Specifically, the sorting unit 133c rearranges blocks of AF555_CD7 in accordance with an arrangement order of blocks in which the positive cell rate of AF488_CD3 is in descending order. In addition, the sorting unit 133c rearranges blocks of AF555_CD5 in accordance with an arrangement order of blocks in which the positive cell rate of AF647_CD3 is in descending order.

At the time of the above sorting, the sorting unit 133c sorts blocks on the basis of the block name (for example, 1 of 1 band, 2 of 1 band, 3 of 1 band, . . . ). After the rearrangement, the block names (blocks) are arranged in the same order in AF555_CD3 and AF647_CD7. The same applies to AF555_CD3 and AF647_CD5, and block names (blocks) are arranged in the same order after sorting.

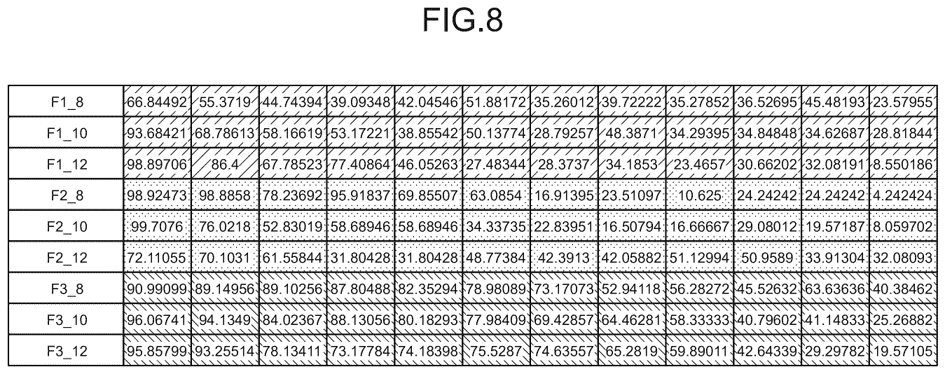

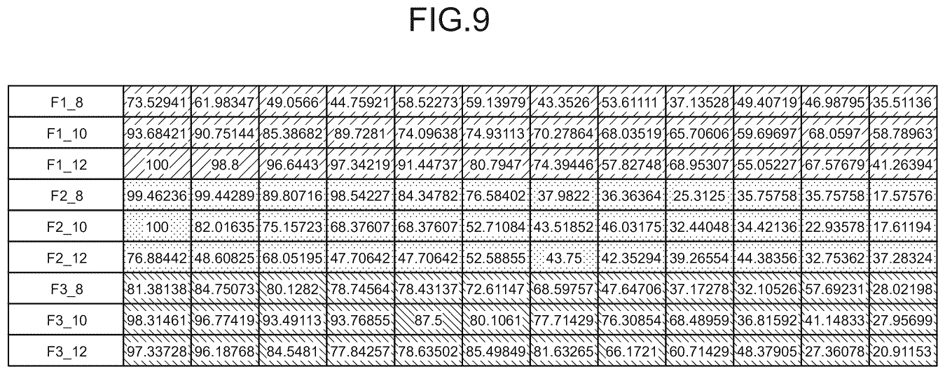

FIG. 8 is a diagram illustrating an example of the positive cell rate for each block after sorting AF488_CD7 according to the present embodiment. FIG. 9 is a diagram illustrating an example of the positive cell rate for each block after sorting AF647_CD5 according to the present embodiment. As illustrated in FIG. 8, the blocks of AF488_CD7 are arranged in the order of arrangement of blocks in which the positive cell rate of AF555_CD3 is in descending order. Further, as illustrated in FIG. 9, the blocks of AF647_CD5 are also arranged in the order of arrangement of blocks in which the positive cell rate of AF555_CD3 is in descending order.

### (Matrix Decomposition Processing Holding Position Information)

The correlation analysis unit 133d performs matrix decomposition processing on the data of the sorted and rearranged matrix, for example, matrix decomposition processing corresponding to a combination of a plurality of biomarkers as described above. Here, all values are positive cell rates, and thus the matrix normalization is not performed and non-negative value processing is also skipped since all are positive values. For example, the correlation analysis unit 133d processes two matrices by JNMF and performs matrix decomposition (dimensional compression). At this time, the correlation analysis unit 133d performs simultaneous decomposition of the plurality of matrices while holding the position information (space information). Note that the correlation analysis unit 133d acquires information regarding each biomarker and information such as the number of clusters k as input data.

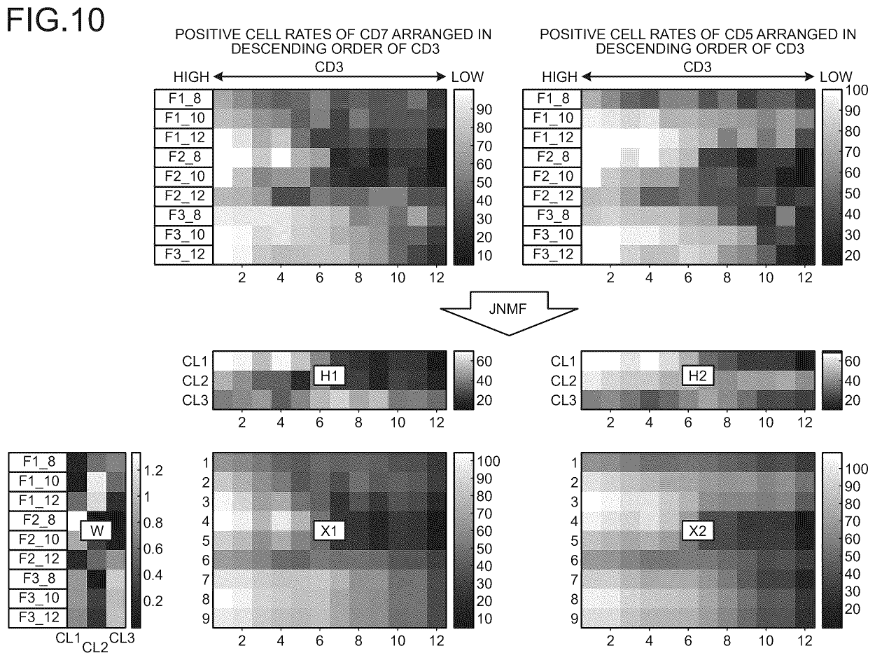

FIG. 10 is a diagram for describing Example 1 of the JNMF according to the present embodiment. In the example of FIG. 10, the number of clusters k is set to k=3, for example, since the field of view is three fields of view. Note that the number of clusters k is appropriately set, but may be obtained by an elbow method or the like. The calculation of the number of clusters k by the elbow method will be described later in detail. Note that, in the example of FIG. 10, CD3 is AF555_CD3, CD5 is AF647_CD5, and CD7 is AF488_CD7. Hereinafter, AF555_CD3 may be referred to as CD3, AF647_CD5 may be referred to as CD5, and AF488_CD7 may be referred to as CD7.

Here, the joint NMF (JNMF) is an extension of the non-negative matrix factorization (NMF). This JNMF can target multiple matrices and enables integrated analysis of multi-omics data. NMF is to decompose one matrix into two small matrices. Here, a certain matrix is an N×M matrix X, and the matrix X can be expressed as a product of the matrices W and H. Specifically, the NMF decomposes the matrix X of non-negative N rows and M columns (N×M) into a matrix W of non-negative N rows and k columns (N×k) and a matrix H of non-negative k rows and M columns (k×M) (X=WH). For example, the matrix W and the matrix H are determined in such a manner that a mean square residual D between the matrix X and the product (W*H) of the matrix W and the matrix H is minimized. k is the number of clusters. Note that the NMF is a technique suitable for emphasizing the relevance between matrix elements by decomposition of potential elements instead of explicit clustering, and further for capturing outliers such as mutation and overexpression.

Note that, as a method of matrix decomposition processing, infinite NMF (INMF), multiple canonical correlation analysis (MCCA), multi-block partial least-squares (MB-PLS), joint and individual variation explained (JIVE), and the like can be used in addition to the JNMF described above.

Here, in the example of FIG. 10, three classes (clusters) are CL1, CL2, and CL3. CL1 is the first column of W and the first row of H1 and H2. CL2 is the second column of W and the second row of H1 and H2. CL3 is the third column of W and the third row of H1 and H2. Here, data is divided into a common basis vector W and feature vectors H1 and H2.

### (Clustering Processing)

The correlation analysis unit 133d classifies the samples into each cluster on the basis of the value of the common basis vector W, and determines the membership (clustering). In determining the membership for each cluster, a region whose value is equal to or more than a threshold may be determined as the membership of the cluster, or the membership of the cluster may be obtained from a Z score.

### (Extraction of Common Module)

The correlation analysis unit 133d extracts a region (block) having a high value of the feature vector for each cluster as a membership of a common module. For example, the correlation analysis unit 133d extracts a feature amount (for example, a positive rate) of a cell for each common module on the basis of a correlation of each biomarker, that is, the membership of the common module for each cluster. The common module is a region extracted as a membership related to a clustering result. The membership is a component extracted as a common feature amount related to a clustering result, and is, for example, a constituent region (for example, regions, blocks, or the like) extracted as the common feature amount.

In determining the membership of the common module, a region whose value is equal to or more than a threshold may be determined as the membership of the common module, or the membership of the common module may be determined from a Z score. Note that a method of obtaining the membership of the common module from the Z score will be described later in detail.

In the example of FIG. 10, CL1 includes the field of view F3 in a main region of the field of view F2, but as the membership of a common module of CL1, a region in which CD3 is high and CD7 is high and CD3 is high and CD5 is high in the region of the field of view F2 is extracted. In CL2, the field of view F1 is classified, and as the membership of the common module, a region in which CD3 is high and CD7 is high and CD3 is high and CD5 is high in the region of the field of view F1 is extracted. In addition, as CL3, a region of the field of view F3 is classified. The feature amount (for example, the positive rate) of the cell for each common module is extracted on the basis of the classification of the sample for each cluster.

As in the above results, clusters can be divided for each field of view (F1, F2, or F3) due to slight differences in positive cell rate. In addition, a region in which CD3 is high and CD7 is high and CD3 is high and CD5 is high can be extracted as having a correlation. Note that, CD3, CD5, and CD7 are markers of T cells, and thus results similar to expected results could be obtained.

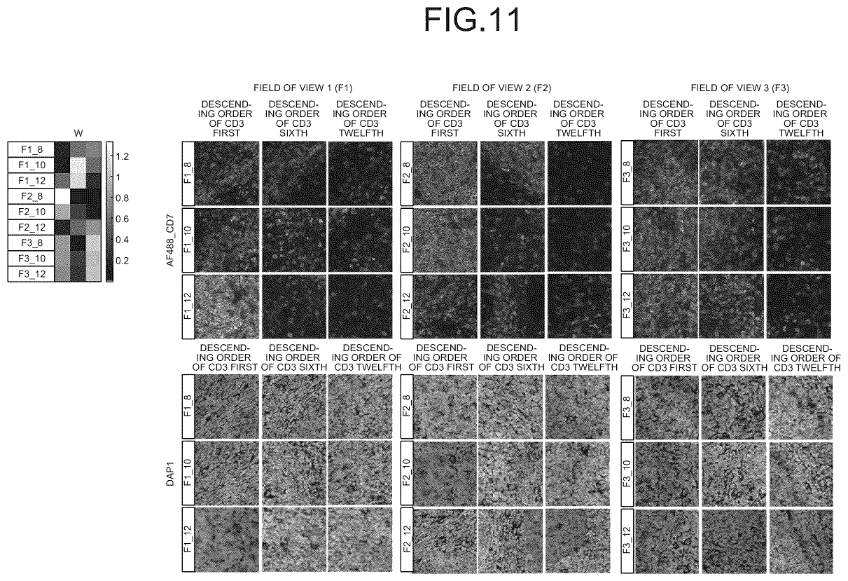

Here, FIG. 11 is a diagram for describing a comparison between a classification result of W according to the present embodiment and an actual image. Note that, in the example of FIG. 11, CD3 is AF555_CD3.

As illustrated in FIG. 11, a region having a high value for each CL of W in the JNMF was compared with visual evaluation of an actual image. The actual image was compared with the block having the first highest value, the block having the sixth highest value (median value), and the block having the twelfth highest value (minimum value) when sorted in descending order of CD3 for each region. Since CD7 and CD5 have similar tendencies, only CD7 images are illustrated this time.

For each field of view, the images were compared with the positive cell rate of each biomarker and results of the JNMF. Although the field of view F1 (field of view 1) is mainly assigned as CL2, only F1_8 had a CD5 positive rate of 73.5% in the region having the highest value when sorted by CD3, whereas both F1_10 and F1_12 had a CD5 positive rate exceeding 90%. This is reflected in the result in which the value of CL2 of W is low only for F1_8. In addition, although the field of view F2 (field of view 2) is mainly assigned as CL1, in the region having the highest value when sorted by CD3, only F2_12 was in the 70% range for both of CD5 and 7, whereas F2_8 and F2_10 were both more than 95%. This is reflected in the result in which the value of CL1 of W is lower only for F2_12. In addition, although the field of view 3 (F3) is assigned to CL3, F3_8, F3_10, and F3_12 showed similar tendencies in CD5 and CD7. This is reflected in the result that the value of CL3 of W was high in all of F3_8, F3_10, and F3_12. From these, it can be seen that the JNMF can execute clustering by reflecting fine feature amounts.

According to the correlation analysis of such a series of multi-biomarkers, it is possible to analyze the interaction between the position information and the positive cells of the multi-biomarkers by performing clustering processing on specimen images stained with a plurality of biomarkers while holding the position information. That is, the correlation between different biomarkers can be analyzed and acquired from the positive rate and position information of different biomarkers.

Note that, in the above description, three fields of view are designated from one specimen, but it is not limited thereto, and by designating a plurality of fields of view in a wider region, the feature amount of the cell for each field of view can be extracted even in the same specimen. In addition, it is also possible to perform comparison for each specimen using a whole slide of the specimen, and the comparison result can be applied to patient selection. In addition, by using different specimens as samples (for example, tonsils, lymph, large intestine, bone marrow, skin, and the like), it is possible to examine the correlation between the common feature amount and the multi-marker of different cancer cells, and the result can be applied to drug prediction of the type of cancer, and the like.

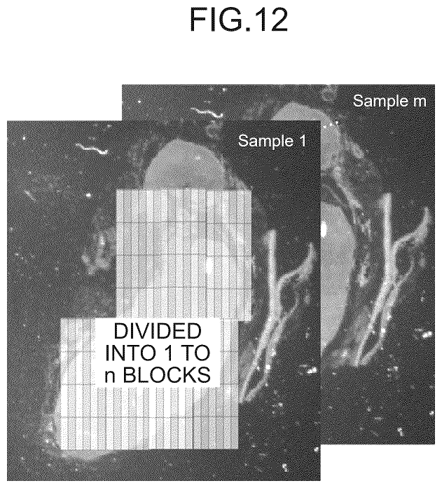

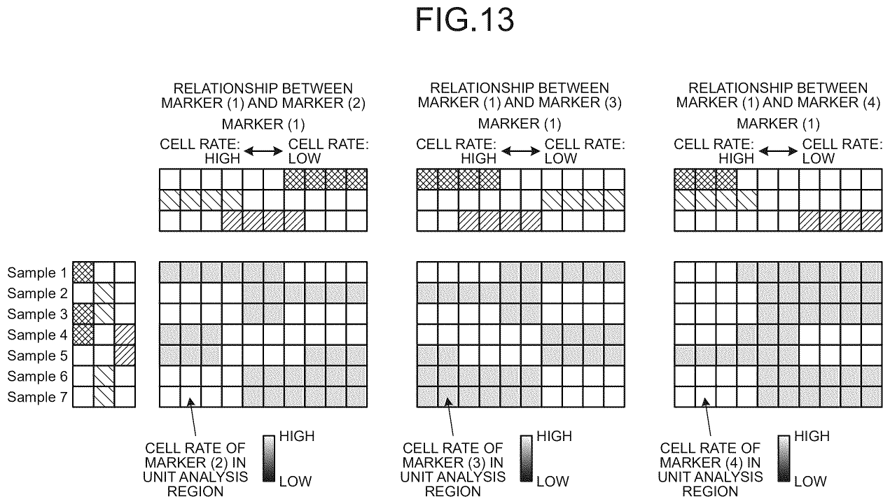

FIG. 12 is a diagram for describing Example 2 of samples according to the present embodiment (when the field of view is selected for each specimen). FIG. 13 is a diagram for describing Example 2 of the JNMF according to the present embodiment. As illustrated in FIG. 12, for example, a field of view is set for each specimen (for example, samples 1 to m), and the field of view is divided into 1 to n blocks. Note that the number of blocks may be settable by an input operation of the user on the operating unit 160.

Next, as illustrated in FIG. 13, the space analysis unit 133 executes necessary processing such as sorting processing on the field of view of the specimen for each specimen (for example, samples 1 to 7). For example, the sorting unit 133c executes sorting processing on a biomarker (2), a biomarker (3), and a biomarker (4) on the basis of a biomarker (1). In this manner, it is also possible to perform correlation analysis by performing sorting for each specimen.

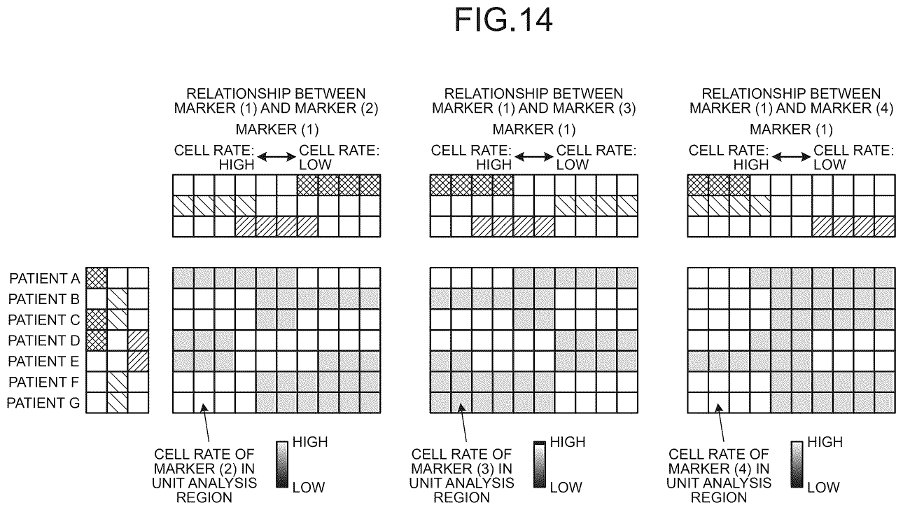

FIG. 14 is a diagram for describing Example 3 of the JNMF according to the present embodiment. As illustrated in FIG. 14, the space analysis unit 133 executes necessary processing such as sorting processing on a specimen of a patient for each patient (for example, patients A to G) of the specimen. For example, the sorting unit 133c executes sorting processing on the biomarker (2), the biomarker (3), and the biomarker (4) on the basis of the biomarker (1), as in Example 2 of FIG. 13. In this manner, it is also possible to perform correlation analysis by performing sorting for each patient of the specimen.

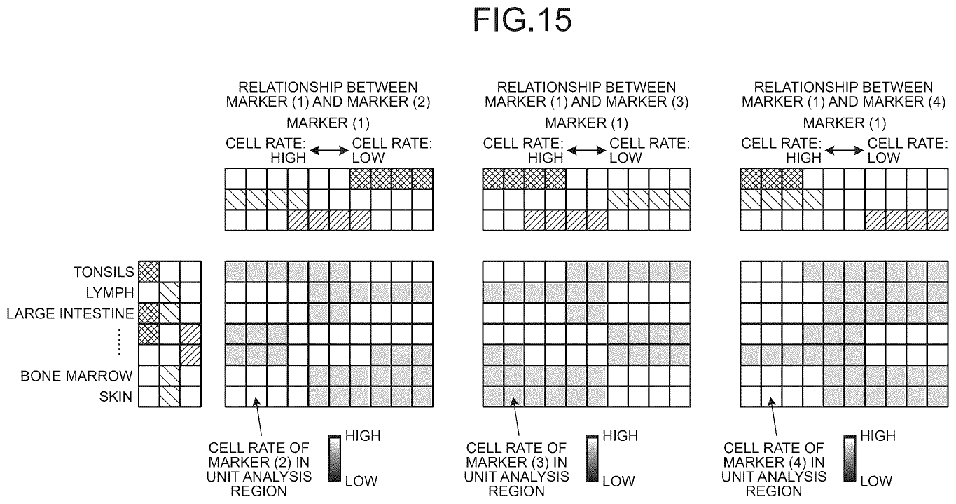

FIG. 15 is a diagram for describing Example 4 of the JNMF according to the present embodiment. As illustrated in FIG. 15, the space analysis unit 133 executes necessary processing such as sorting processing on the specimen of the patient site for each patient site (for example, tonsils, lymph, large intestine, bone marrow, skin, and the like) of the specimen. For example, the sorting unit 133c executes sorting processing on the biomarker (2), the biomarker (3), and the biomarker (4) on the basis of the biomarker (1), as in Example 2 of FIG. 13. In this manner, it is also possible to perform correlation analysis by performing sorting for each patient part of the specimen.

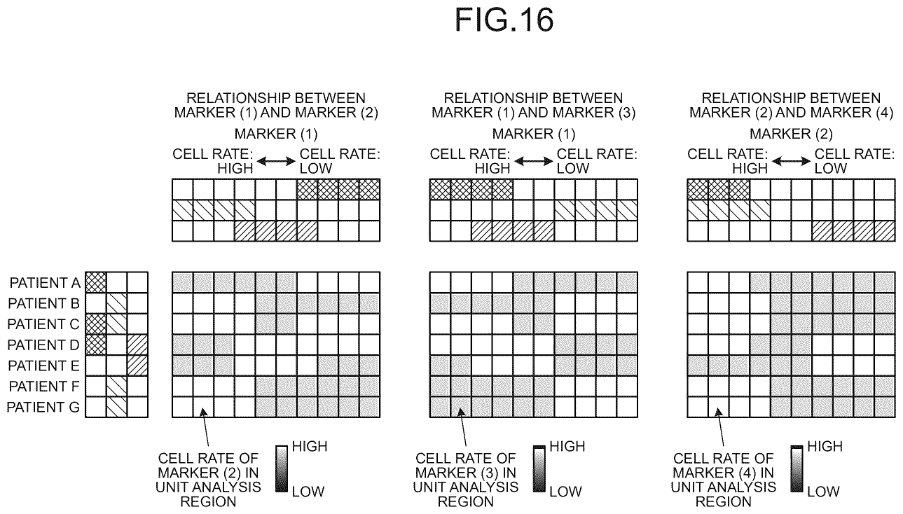

FIG. 16 is a diagram for describing Example 5 of the JNMF according to the present embodiment. As illustrated in FIG. 16, the space analysis unit 133 executes necessary processing such as sorting processing and clustering processing on a specimen of a patient for each patient (for example, patients A to G) of the specimen, as in Example 3 of FIG. 14. However, the sorting unit 133c executes sorting processing on the biomarkers (2) and (3) on the basis of the biomarkers (1), and executes sorting processing on the biomarkers (4) on the basis of the biomarkers (2). As described above, it is also possible to perform correlation analysis by performing sorting using a plurality of types of markers instead of using one biomarker used for sorting.

### (Determination of Number of Clusters)

The correlation analysis unit 133d can determine the number of clusters k from an error tendency of residuals, for example. The correlation analysis unit 133d can obtain a residual sum of squares (SSE) of the JNMF while changing the number of clusters k, and obtain the optimum number of clusters k from the change tendency of the residual sum of squares. Note that, in a case where it is difficult to recognize the change tendency when obtaining the optimum number of clusters k, the optimum number of clusters k can be further obtained by a method such as an elbow method. The elbow method is a method of finding a combination in which both the SSE and the number of clusters k are as small as possible. Note that, for example, the number of clusters k that minimizes the residual or Euclidean distance may be set, or the number of clusters may be set as desired by the user. That is, the number of clusters k may be settable by an input operation of the user on the operating unit 160.

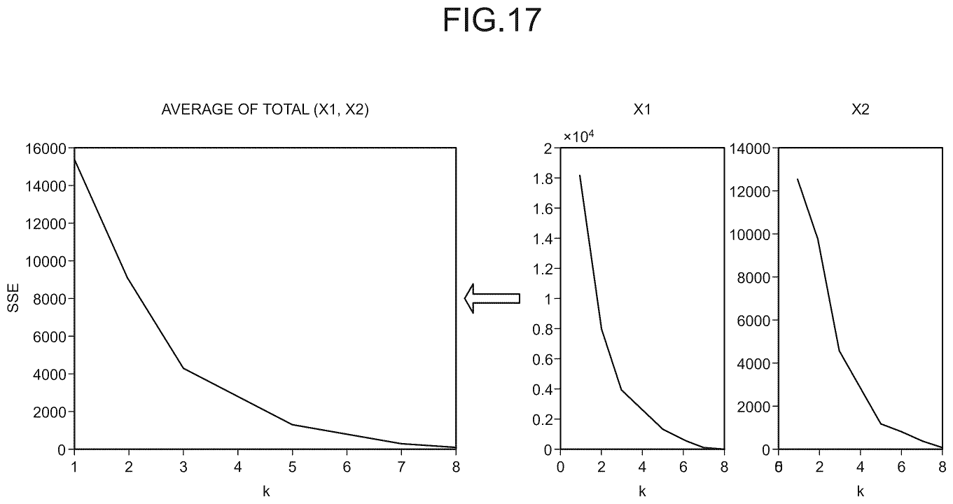

Here, FIG. 17 is a diagram for describing determination of the number of clusters k according to the present embodiment. In the example of FIG. 17, the vertical axis represents SSE, and the horizontal axis represents the number of clusters k. As illustrated in FIG. 17, the correlation analysis unit 133d obtains the SSE of the JNMF for the matrix X1 and the SSE of the JNMF for the matrix X2 while changing the number of clusters k, and obtains the average of the whole (X1, X2). From the relationship information between the number of clusters k and the SSE, for example, a combination in which the SSE is about 4000 and the number of clusters k is 3 is selected as a combination in which both the SSE and the number of clusters k are as small as possible. Thus, the number of clusters k is determined to be three.

### (Determination of Cluster Membership and Common Module Membership)

The correlation analysis unit 133d can set a cluster from the maximum value in a case where it is always desired to assign a sample to one cluster for each sample or space. However, depending on the sample, it is conceivable that the sample belongs to a plurality of clusters or does not belong to all clusters, and thus, it is possible to obtain the membership of the cluster from the Z score.

For example, the correlation analysis unit 133d calculates the Z score (Zᵢⱼ) of each element of each column of W and each row of H using the relational expression of Zᵢⱼ=(Xᵢⱼ−Uᵢ)/σᵢ. Here, Uᵢ is an average or median value of the positive cell rate, the number of positive cells, the luminance value, or the like of each biomarker in Hi (i=1, 2, 3, . . . ). σᵢ is a standard deviation or a median absolute deviation.

If Zᵢⱼ is larger than the threshold T, the correlation analysis unit 133d assigns Zᵢⱼ as the membership of the common module. The threshold T is set in advance. The threshold T may be set to a value equal to or more than 2 from statistical superiority, or may be set to a value more suitable for the user from a membership tendency of the cluster. The threshold T may be settable by an input operation of the user on the operating unit 160.

Note that, as an expression of feature amount extraction other than the Z score, it is also possible to use feature amount=Xᵢⱼ−Uᵢ (difference from average) or feature amount=Xᵢⱼ−Uᵢ/Uᵢ (not divided by standard deviation, but divided by an average value).

### (Confirmation of Correlation of Membership Assignment of Cluster)

In order to evaluate the stability of clustering, the correlation analysis unit 133d may perform correlation analysis using a Pearson's correlation coefficient, pairwise correlation analysis, or the like in order to confirm whether or not features of processing results of each clustering processing are correlated.

### (Cloud Cooperation)

Various types of processing such as a correlation analysis process of multi-biomarkers and a prediction process of drug effect (for example, field of view selection processing, positive cell amount calculation processing, sorting processing, clustering processing, and the like) can be executed by software on the cloud side by performing various types of processing on the cloud side in cooperation with various types of data.

Note that, in the embodiment described above, three or four types of biomarkers are used, but it is not limited thereto, and two or five or more types of biomarkers may be used. In addition, the biomarker used for sorting may be, for example, an immune cell marker or a tumor marker. Note that examples of the biomarkers include molecular biomarkers and cell biomarkers.

### <1-5. Operation and Effect>

As described above, according to the present embodiment, the information processing device 100 includes the acquisition unit 110 that acquires a fluorescence spectrum derived from a biological sample and position information of the biological sample from a sample including the biological sample, the identification unit 133b that identifies, from the fluorescence spectrum, information regarding a plurality of different biomarkers of the biological sample associated with the position information of the biological sample, and the correlation analysis unit 133d that performs matrix decomposition processing (for example, dimensional compression with position information of multi-biomarkers) corresponding to a combination of the plurality of biomarkers on the information regarding the plurality of biomarkers and outputs a correlation of the information regarding the plurality of biomarkers. This makes it possible to acquire a correlation of information regarding a plurality of biomarkers, so that a correlation of a plurality of biomarkers can be obtained.

Furthermore, the correlation analysis unit 133d may perform the matrix decomposition processing by the JNMF on the information regarding the plurality of biomarkers, and then perform the clustering processing. This makes it possible to reliably obtain a correlation of a plurality of biomarkers.

Furthermore, the correlation analysis unit 133d may obtain the residual sum of squares (SSE) of the JNMF while changing the number of clusters k in the clustering processing, and determine the number of clusters k from the change tendency of the residual sum of squares. Thus, the appropriate number of clusters k can be obtained.

In addition, the number of clusters k in the clustering processing may be set by the user. Thus, the number of clusters k desired by the user can be set.

Furthermore, the information processing device 100 may further include the selection unit 133a that determines a predetermined region (for example, the field of view F1, the field of view F2, and the field of view F3) of the sample, and the identification unit 133b may identify, from the fluorescence spectrum of the predetermined region, information regarding a plurality of biomarkers associated with the position information of the biological sample of the predetermined region. Thus, a correlation between biomarkers in a predetermined region (for example, a region of interest) of the sample can be obtained.

Furthermore, the selection unit 133a may determine a plurality of predetermined regions (for example, the field of view F1, the field of view F2, and the field of view F3). Thus, a correlation of each biomarker in a plurality of predetermined regions of the sample can be obtained.

In addition, the number of clusters k in the clustering processing may be set according to the number of predetermined regions. This makes it possible to reliably obtain the correlation between the biomarkers in a plurality of predetermined regions of the sample.

Further, the predetermined region may be set by the user. Thus, it is possible to set a predetermined region desired by the user, and hence it is possible to obtain a correlation between biomarkers in the predetermined region according to a desire of the user.

Furthermore, the selection unit 133a may determine the predetermined region (for example, the field of view F1, the field of view F2, and the field of view F3) of the common position of the plurality of samples, the acquisition unit 110 may acquire the fluorescence spectrum and the position information of the biological sample for each predetermined region, the identification unit 133b may identify, from the fluorescence spectrum for each of the predetermined regions, information regarding the plurality of biomarkers for each of the predetermined regions associated with position information of the biological sample for each of the predetermined regions, and the correlation analysis unit 133d may perform the matrix decomposition processing on the information regarding the plurality of biomarkers for each of the predetermined regions, and output a correlation of the information regarding the plurality of biomarkers for each of the predetermined regions. Thus, the correlation of the biomarkers in the predetermined region at the common position of the plurality of samples can be obtained.

Furthermore, the selection unit 133a may determine the predetermined region (for example, the field of view F1, the field of view F2, and the field of view F3) of different positions of the plurality of samples, the acquisition unit 110 may acquire the fluorescence spectrum and the position information of the biological sample for each predetermined region, the identification unit 133b may identify, from the fluorescence spectrum for each predetermined region, information regarding the plurality of biomarkers for each of the predetermined regions associated with position information of the biological sample for each of the predetermined regions, and the correlation analysis unit 133d may perform the matrix decomposition processing on the information regarding the plurality of biomarkers for each predetermined region, and output a correlation of the information regarding the plurality of biomarkers for each of the predetermined regions. Thus, the correlation of the biomarkers in the predetermined region at different positions of the plurality of samples can be obtained.

In addition, the plurality of samples may be a plurality of different specimens. Thus, the correlation of the biomarkers in the plurality of different specimens can be obtained.

Furthermore, the plurality of specimens may be specimens for each of patients. Thus, the correlation of the biomarkers in the specimen for each patient can be obtained.

In addition, the plurality of specimens may be specimens for each part of the patient. Thus, the correlation of the biomarkers in the specimen for each part of the patient can be obtained.

Furthermore, the information processing device 100 may further include the sorting unit 133c that changes, on the basis of the arrangement order of the plurality of pieces of unit information (for example, block) included in the information regarding one biomarker among the information regarding the plurality of biomarkers, an arrangement order of a plurality of pieces of unit information (for example, blocks) included in information regarding other biomarkers, and the correlation analysis unit 133d may perform the matrix decomposition processing on the information regarding the plurality of biomarkers in which the arrangement order has been changed, and output a correlation of the information regarding the plurality of biomarkers. This makes it possible to reliably obtain the correlation of the plurality of biomarkers.

Furthermore, the information processing device 100 may further include an information acquisition unit 111 that acquires an administration drug candidate for a patient related to a biological sample, and an estimation unit 133e that estimates efficacy of the administration drug candidate to the patient from a correlation of information regarding the plurality of biomarkers and the administration drug candidate for the patient. This makes it possible to estimate the efficacy of the administration drug candidate to the patient.

Furthermore, the estimation unit 133e may extract a membership of a common module from the correlation of the information regarding the plurality of biomarkers, and estimate the efficacy of the administration drug candidate to the patient from the membership of the common module and the administration drug candidate for the patient. This makes it possible to reliably estimate the efficacy of the administration drug candidate to the patient.

Further, the information regarding the biomarkers may also be the degree of positive cells (for example, the amount of positive cells). This makes it possible to reliably obtain the correlation of the plurality of biomarkers.

In addition, the information regarding the biomarker may be a positive cell rate indicating the degree of positive cells, the number of positive cells, or a luminance value. This makes it possible to reliably obtain the correlation of the plurality of biomarkers.

### 2. Second Embodiment

### 2-1. Summary of Second Embodiment

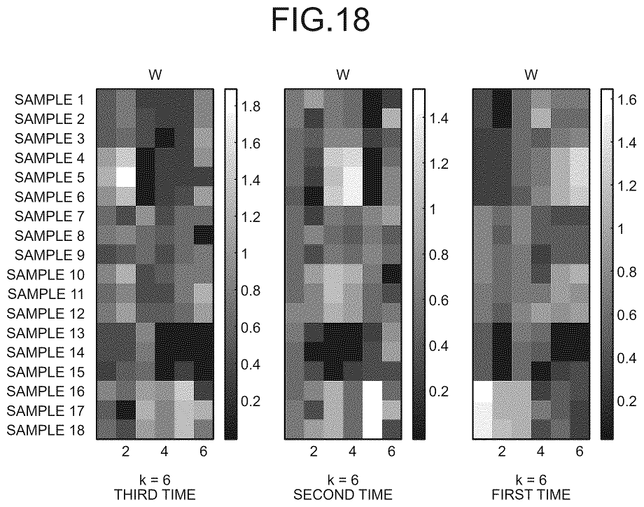

The gist of a second embodiment according to the present embodiment will be described with reference to FIG. 18. FIG. 18 is a diagram illustrating a result W of executing the JNMF (jNMF) according to the present embodiment a plurality of times.

In step S29 according to the first embodiment described above, initial values are given to W, H1, H2, . . . , and Hn to perform calculation at the beginning of calculation of the JNMF. By default, for example, a random number is assigned to initial values of W, H1, H2, . . . , and Hn. Because the initial value becomes random by giving a random number to the initial value, the result may vary. In particular, as the number of clusters increases, the possibility that the results vary increases.

FIG. 18 illustrates a result W of executing the JNMF a plurality of times in a case where the number of clusters k=6. Specifically, a result W of executing the JNMF a plurality of times with the number of clusters k=6 using a simulation data set of 18 samples is illustrated. The number of executions is three (first, second, and third). As illustrated in FIG. 18, the initial value is different for each execution count, and thus the result W is different for each execution count. As described above, the result W may vary.

Accordingly, in the second embodiment, an appropriate predetermined initial value (for example, an optimum predetermined initial value) is given to the initial value instead of the random number. The predetermined initial value is, for example, a predetermined fixed value. This stabilizes the results. In addition, clustering can be performed on the basis of an existing JNMF clustering result. Furthermore, by giving an appropriate predetermined initial value to the initial value, the calculation time can be significantly shortened as compared with a case where a random number is used. The calculation result converges quickly, and thus the loop number, the iteration number, and the like of the JNMF are smaller than those in a case where a random number is used.

### <2-2. Processing Example of Initial Value Calculation>

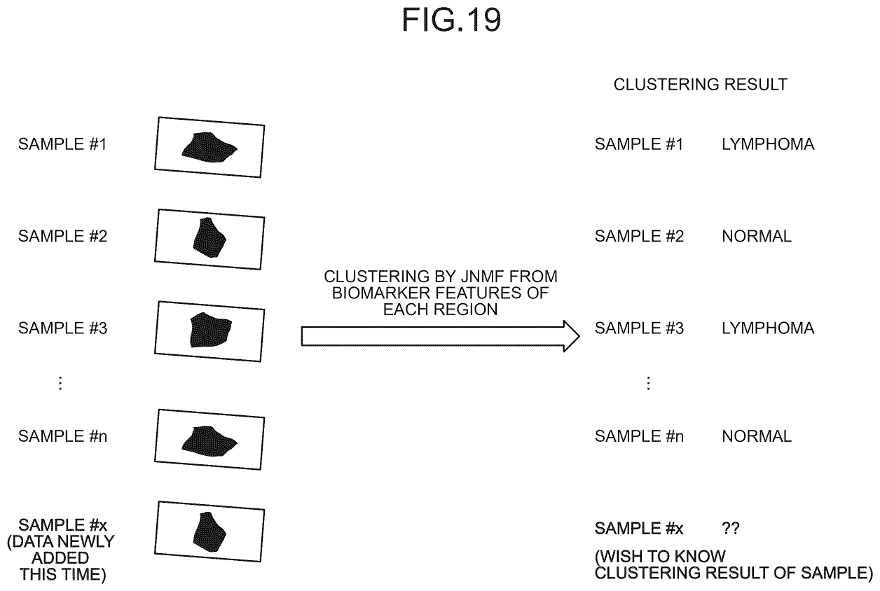

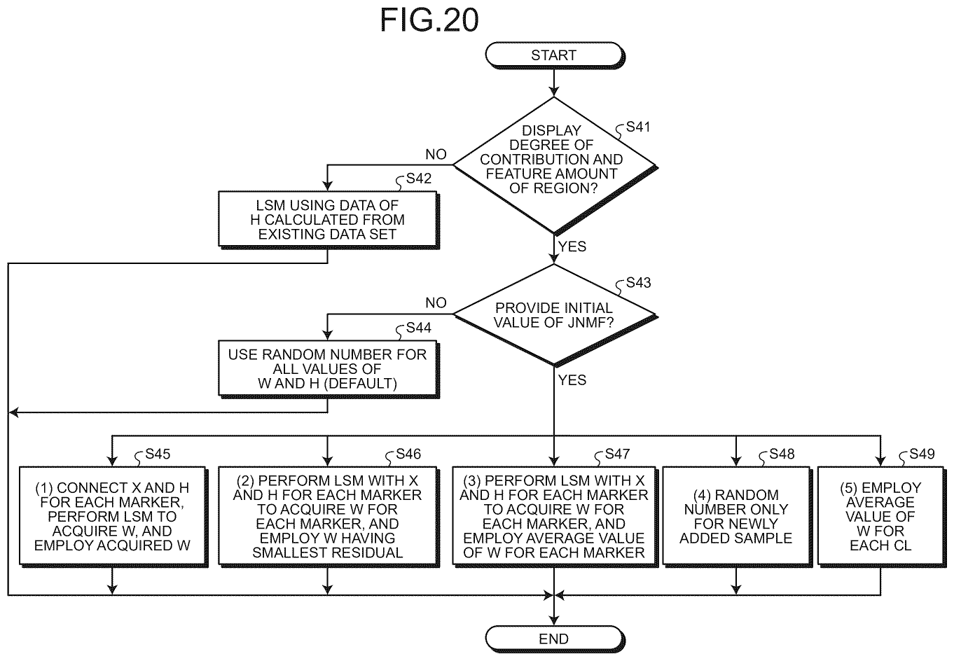

A processing example of initial value calculation according to the present embodiment will be described with reference to FIGS. 19 and 20. FIG. 19 is a diagram for describing an image of clustering according to the present embodiment. FIG. 20 is a flowchart illustrating an example of a flow of processing of initial value calculation according to the present embodiment.

As illustrated in FIG. 19, there is already a simulation data set (for example, sample #1 to sample #n) in which the JNMF is executed. One or more samples (for example, sample #x) are newly added to this data set, and the JNMF is executed to obtain a clustering result. In such a situation, in order to implement the JNMF using a predetermined initial value, it is necessary to obtain the predetermined initial value in advance. A processing example of initial value calculation for obtaining the predetermined initial value will be described below.

As illustrated in FIG. 20, in step S41, the correlation analysis unit 133d determines whether or not to display the degree of contribution and the feature amount of the region. In step S42, the correlation analysis unit 133d performs LSM using the data of H calculated from the existing data set. In step S43, the correlation analysis unit 133d determines whether or not to provide an initial value of the JNMF. In step S44, the correlation analysis unit 133d uses random numbers for all the values of W and H (default).

In step S45, X and H of each marker are connected, LSM is performed with X and H of each connected marker to acquire W, and the acquired W is employed as a predetermined initial value (first calculation method). In step S46, LSM is performed with X and H for each marker to acquire W for each marker, and W having a smallest residual is employed as a predetermined initial value from the acquired W for each marker (second calculation method). In step S47, LSM is performed with X and H for each marker to acquire W for each marker, and an acquired average value of W for each marker is employed as a predetermined initial value (third calculation method). In step S48, a random number is employed as a predetermined initial value only for the newly added sample (fourth calculation method). In step S49, the average value of W for each CL is employed as a predetermined initial value (fifth calculation method). The first to fifth calculation methods according to steps S45 to S49 described above will be described in detail later.

Note that each step in the flowchart of FIG. 20 is not necessarily processed in time series in the described order. That is, each step in the flowchart may be processed in an order different from the described order or may be processed in parallel. In addition, the information processing device 100 may also execute processing not illustrated in FIG. 20.

### <2-3. Example of Initial Value Calculation>

### <2-3-1. Simulation Data Set>

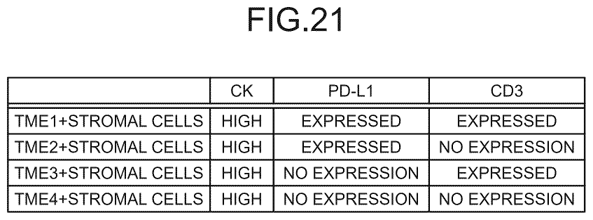

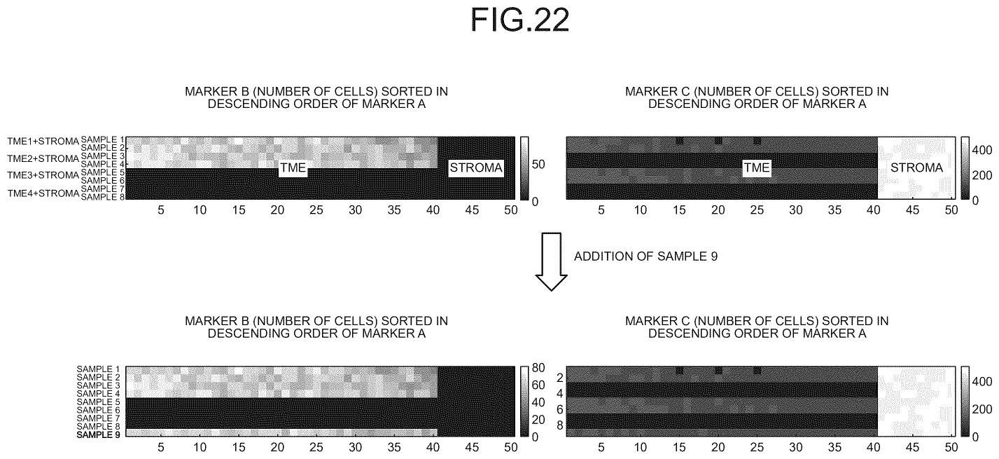

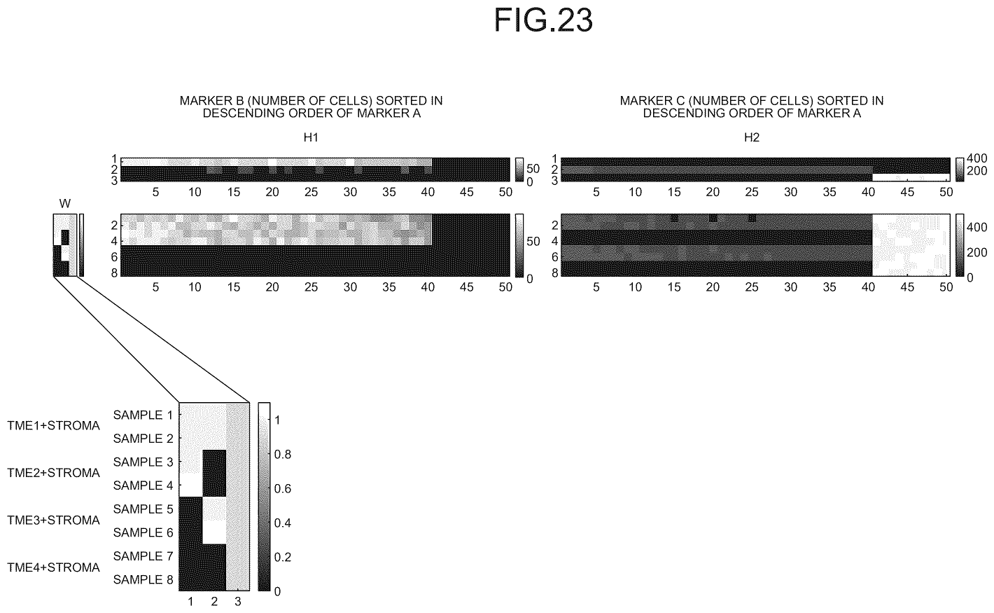

A simulation data set according to the present embodiment will be described with reference to FIGS. 21 to 23. FIG. 21 is a diagram for describing types of cancer microenvironment (TME) according to the present embodiment. FIG. 22 is a diagram for describing a simulation data set according to the present embodiment. FIG. 23 is a diagram for describing a result of executing the JNMF on the samples 1 to 8 according to the present embodiment.

As illustrated in FIG. 21, four types of cancer microenvironment (TME) (presence or absence of infiltration of T cells in tumor/presence or absence of PD-L1) are assumed. Assumed markers are tumor cell markers (CK), lymphocyte markers (CD3), and inhibitor markers (PD-L1). There are tumor and stromal regions.

TME1+stromal cells are a cancer microenvironment of CK (high), PD-L1 (expressed), CD3 (expressed)+stroma. TME2+stromal cells are a cancer microenvironment of CK (high), PD-L1 (expressed), CD3 (no expression)+stroma. TME3+stromal cells are a cancer microenvironment of CK (high), PD-L1 (no expression), CD3 (expressed)+stroma. TME4+stromal cells are a cancer microenvironment of CK (high), PD-L1 (no expression), CD3 (no expression)+stroma.

Simulation data simulating four types of such cancer microenvironments was created. Specifically, simulation data was created assuming that regions of four types of cancer microenvironments were selected as ROIs (regions of interest). Note that, assuming that the region is divided by the ROI, the tumor cell marker is almost 100%. In addition, the number of positive cells is counted for each marker, and sorting is performed with a marker of CK (positive number). For example, descending order sorting is performed.

As illustrated in FIG. 22, first, two samples were created for each of the above-described four types of TMEs, and a simulation data set of eight samples in total was created (samples 1 to 8). Note that, in the example of FIG. 22, a marker A, which is a reference marker, is CK, a marker B is PD-L1, and a marker C is CD3. It is assumed that the number of positive cells is counted.

Samples 1 to 2 were TME1+stromal cells, samples 3 to 4 were TME2+stromal cells, samples 5 to 6 were TME3+stromal cells, and samples 7 to 8 were TME4+stromal cells, and simulation data sets of these samples 1 to 8 were created. Note that the entire region of one sample is assumed to be 50 small regions in total, a region having epithelial tissue+no epithelial tissue (a region having stromal cells) is assumed, and TME is assumed to be 80% (40 small regions), and stromal cells is assumed to be 20% (10 small regions).

Next, a new sample 9 was added to the above-described existing samples 1 to 8 to create a simulation data set of 9 samples in total. In the example of FIG. 22, it is assumed that the samples 1 to 8 are existing simulation data sets and have been subjected to the JNMF, and the sample 9 is added as a specimen to be newly analyzed. The sample 9 is assumed to be TME1+stromal cells.

### (Execution of the JNMF on Existing Samples 1 to 8)

Results of executing the JNMF on the simulation data sets of the existing samples 1 to 8 are illustrated in FIG. 23. In the example of FIG. 23, the JNMF is performed with a default random number as an initial value and the number of clusters k=3. As a result, as illustrated in FIG. 23, CL1 has been clustered with features without expression of PD-L1, CL2 has been clustered with features without infiltration of CD3, and CL3 has been clustered with features of stromal cells.

### <2-3-2. Calculation Methods of Initial Value>

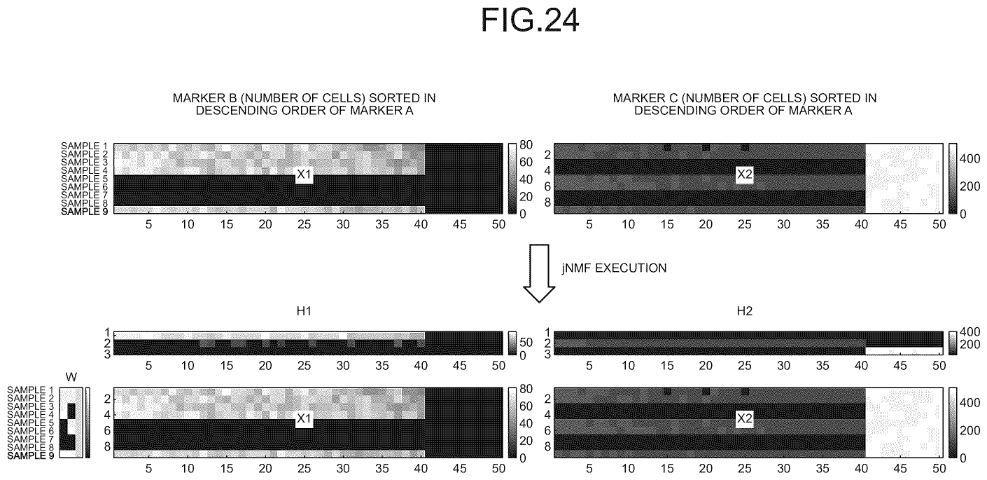

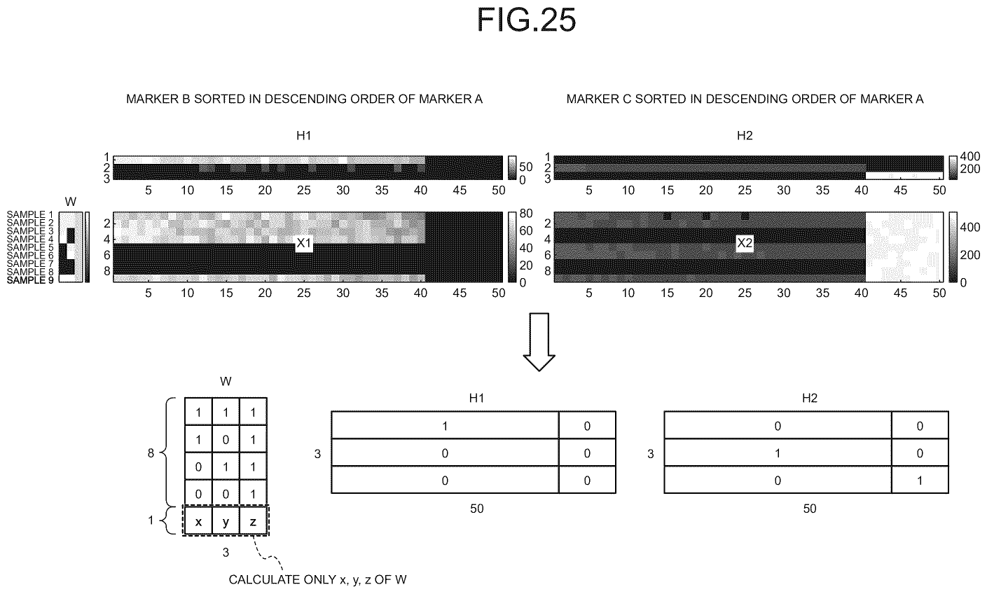

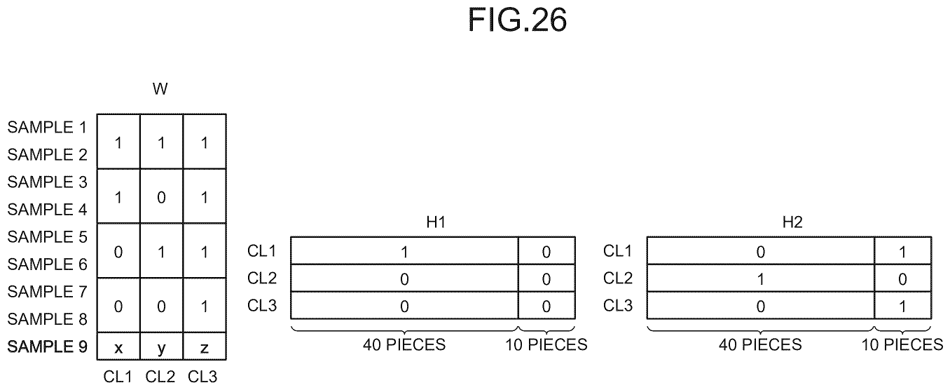

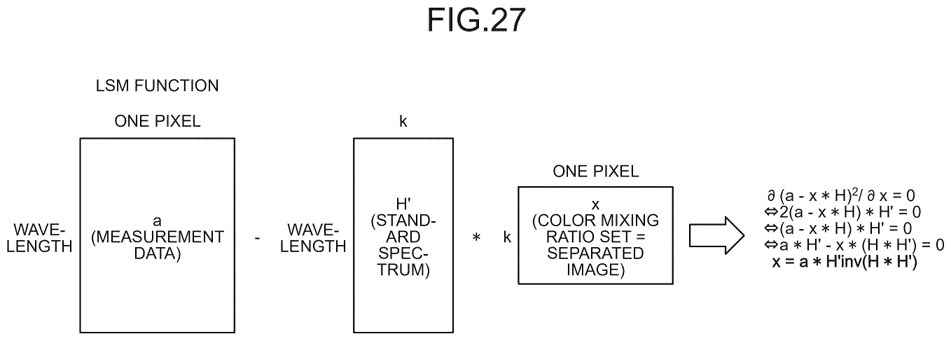

Calculation methods of an initial value according to the present embodiment will be described with reference to FIGS. 24 to 37. FIG. 24 is a diagram for describing that the JNMF is performed on a simulation data set obtained by adding a new sample 9 to the existing samples 1 to 8 according to the present embodiment. FIGS. 25 and 26 are diagrams for describing an outline of an initial value according to the present embodiment. FIG. 27 is a diagram for describing LSM according to the present embodiment.

As illustrated in FIG. 24, the JNMF is performed on the simulation data set obtained by adding the new sample 9 to the existing samples 1 to 8. A method of calculating the initial value of W used in the JNMF will be described.

As illustrated in FIGS. 25 and 26, as the initial value of W of the existing samples 1 to 8, values binarized with 0 or 1 on the basis of the result of the JNMF are used to calculate the initial values x, y, z of W of the sample 9. Examples of a method of calculating x, y, z of W of the sample 9 include the following five types (first to fifth calculation methods). Note that, for the binarized portion, a random number may be used by default, and values of output results calculated in the following five types may be used as they are. In either case, the initial value of W of the samples 1 to 9 is obtained in advance as a fixed value.

Note that, in the example of FIG. 26, individual initial values of the samples 1 to 2 of W are “111” in the order of CL1 to CL3, individual initial values of the samples 3 to 4 of W are “101” in the order of CL1 to CL3, individual initial values of the samples 5 to 6 of W are “011” in the order of CL1 to CL3, individual initial values of the samples 7 to 8 of W are “001” in the order of CL1 to CL3, and initial values of the sample 9 of W are “xyz” in the order of CL1 to CL3.

Further, the initial value of CL1 of H1 is “111 . . . (40) 000 . . . (10)”, the initial value of CL2 of H1 is “000 . . . (40) 000 . . . (10)”, and the initial value of CL3 of H1 is “000 . . . (40) 000 . . . (10)”. The initial value of CL1 of H2 is “000 . . . (40) 111 . . . (10)”, the initial value of CL2 of H2 is “111 . . . (40) 000 . . . (10)”, and the initial value of CL3 of H2 is “000 . . . (40) 111 . . . (10)”.

### (LSM)

Here, a least squares method (LSM) is used to calculate the initial value. As illustrated in FIG. 27, in the LSM function, the measurement data a is fitted to a standard spectrum H to obtain a color mixing ratio set x. x at which the sum of squares of an expression in FIG. 27 becomes minimum is obtained, that is, it becomes minimum when a partial differentiation with respect to the color mixing ratio x is 0. The desired color mixing ratio set (separated image) is obtained from a relational expression of x=a*H′ *inv (H*H′).

### (First Calculation Method)

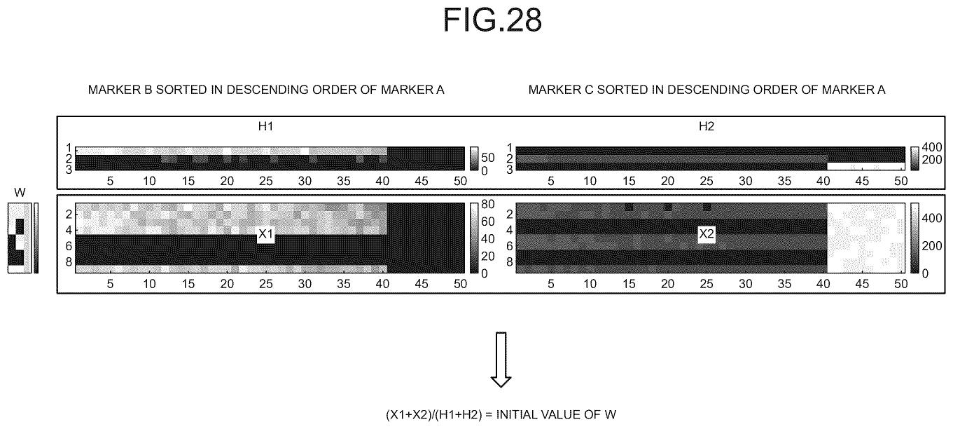

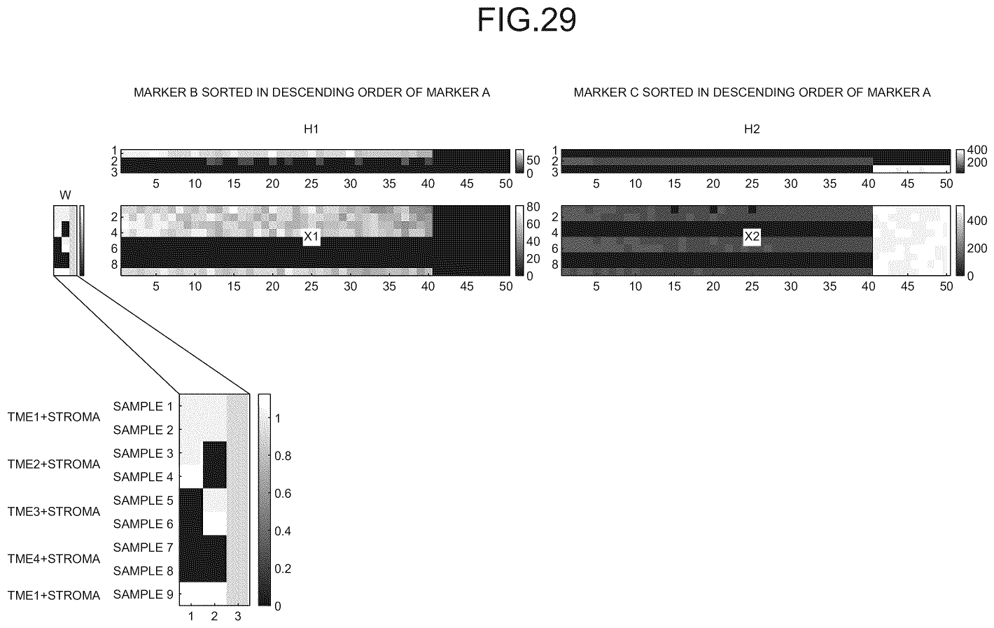

The first calculation method according to the present embodiment will be described with reference to FIGS. 28 and 29. FIG. 28 is a diagram for describing the first calculation method according to the present embodiment. FIG. 29 is a diagram for describing a result of the JNMF using an initial value calculated by the first calculation method according to the present embodiment.

As illustrated in FIG. 28, the correlation analysis unit 133d connects X and H (X1 and H1 of the marker B and X2 and H2 of the marker C) for each marker, performs LSM with X and H for each connected marker to acquire W, and employs the acquired W as a predetermined initial value. For example, the correlation analysis unit 133d uses JNMF calculation results of H1 and H2 of only a data set of the existing samples 1 to 8, performs LSM with X1+X2 and H1+H2, and calculates an initial value of (X1+X2)/(H1+H2)=W. In the example of FIG. 28, the correlation analysis unit 133d connects X1 of the marker B and X2 of the marker C, and connects H1 of the marker B and H2 of the marker C.

Specifically, the correlation analysis unit 133d connects X1 and X2 including the new sample 9, and further connects H1 and H2 calculated by executing the JNMF only with the data set of the existing samples 1 to 8, and performs LSM, thereby calculating the value of W including the new sample 9. The value of W includes the value of xyz of the new sample 9.

Thereafter, the correlation analysis unit 133d gives the calculated initial value to the JNMF, and performs the JNMF on the simulation data set obtained by adding the new sample 9 to the existing samples 1 to 8. Thus, as illustrated in FIG. 29, the samples 1 to 9 are divided into clusters. The initial value of the sample 9 given to the JNMF was considerably close to 1, 1, 1, and the new sample 9 (TME1+stromal cells) segmented into clusters as expected. Note that it is known from a plurality of simulation results that the accuracy of the first calculation method is stably high.

Note that, when LSM is performed with X1+X2 and H1+H2, W is obtained from the relational expression of x=a*H′ *inv (H+H′) (see FIG. 27). In the example of FIG. 27, k corresponds to the number of clusters, x corresponds to H1+H2, a corresponds to X1+X2, and H′ corresponds to w.

### (Second Calculation Method)

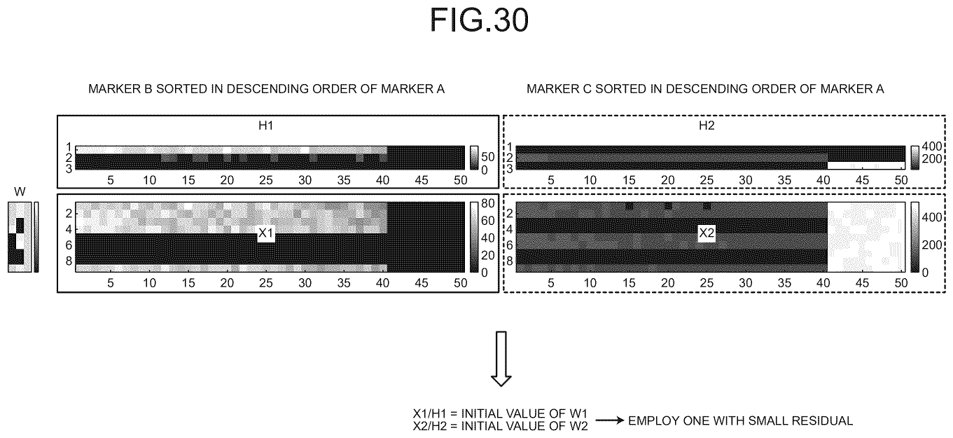

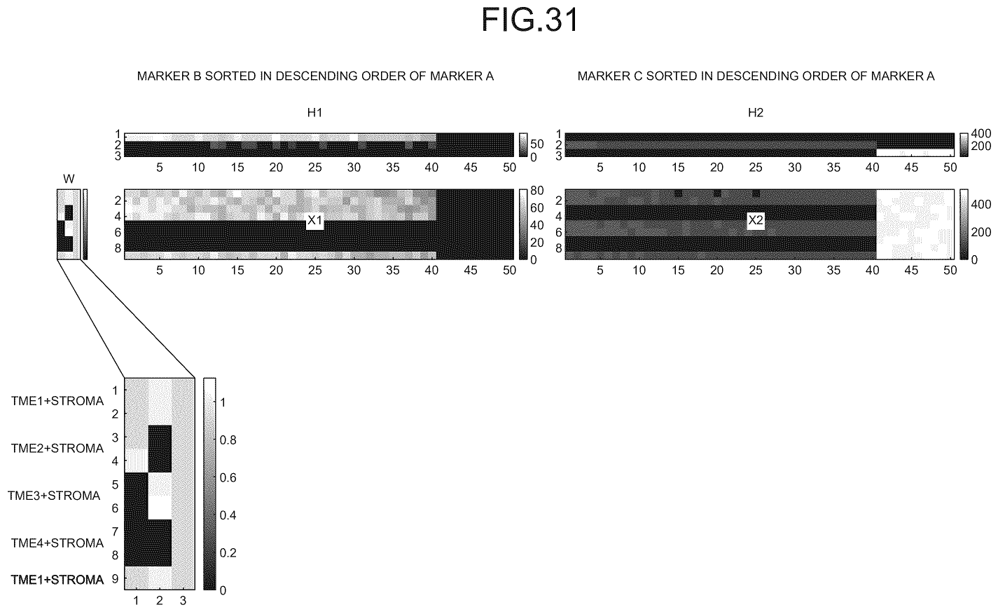

The second calculation method according to the present embodiment will be described with reference to FIGS. 30 and 31. FIG. 30 is a diagram for describing the second calculation method according to the present embodiment. FIG. 31 is a diagram for describing a result of the JNMF using an initial value calculated by the second calculation method according to the present embodiment.

As illustrated in FIG. 30, the correlation analysis unit 133d performs LSM with X and H (X1 and H1 of the marker B and X2 and H2 of the marker C) for each marker to acquire W for each marker, and employs W having the smallest residual from the acquired W for each marker as a predetermined initial value. For example, the correlation analysis unit 133d uses JNMF calculation results of H1 and H2 of only the data set of the existing samples 1 to 8, and employs, as an initial value, one with a smaller residual from the initial value of X1/H1=W1 and the initial value of X2/H2=W2.

Specifically, the correlation analysis unit 133d performs LSM with X1 including the new sample 9 and H1 calculated by executing the JNMF with only the data set of the existing samples 1 to 8, and calculates the value of W1 including the new sample 9. In addition, the correlation analysis unit 133d performs LSM with X2 including the new sample 9 and H2 calculated by executing the JNMF with only the data set of the existing samples 1 to 8, and calculates the value of W2 including the new sample 9. The correlation analysis unit 133d gives the respective values W1 and W2 to the JNMF as initial values, and employs the one having a smaller residual as a predetermined initial value. The value of W1 or W2 includes the value of xyz of the new sample 9.

Thereafter, the correlation analysis unit 133d gives the calculated initial value to the JNMF, and performs the JNMF on the simulation data set obtained by adding the new sample 9 to the existing samples 1 to 8. Thus, as illustrated in FIG. 31, the samples 1 to 9 are divided into clusters. The new sample 9 (TME1+stromal cells) segmented into clusters as expected.

Note that, when LSM is performed with X1 and H1, W is obtained from the relational expression of x=a*H′ *inv (H+H′) (see FIG. 27). In the example of FIG. 27, k corresponds to the number of clusters, x corresponds to H1, a corresponds to X1, and H′ corresponds to w. When LSM is performed with X2 and H2, W is obtained similarly to when LSM is performed with X1 and H1.

### (Third Calculation Method)

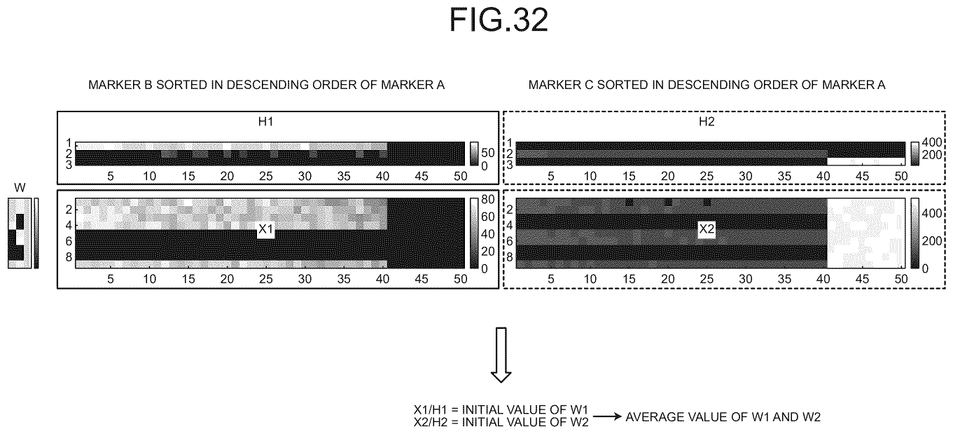

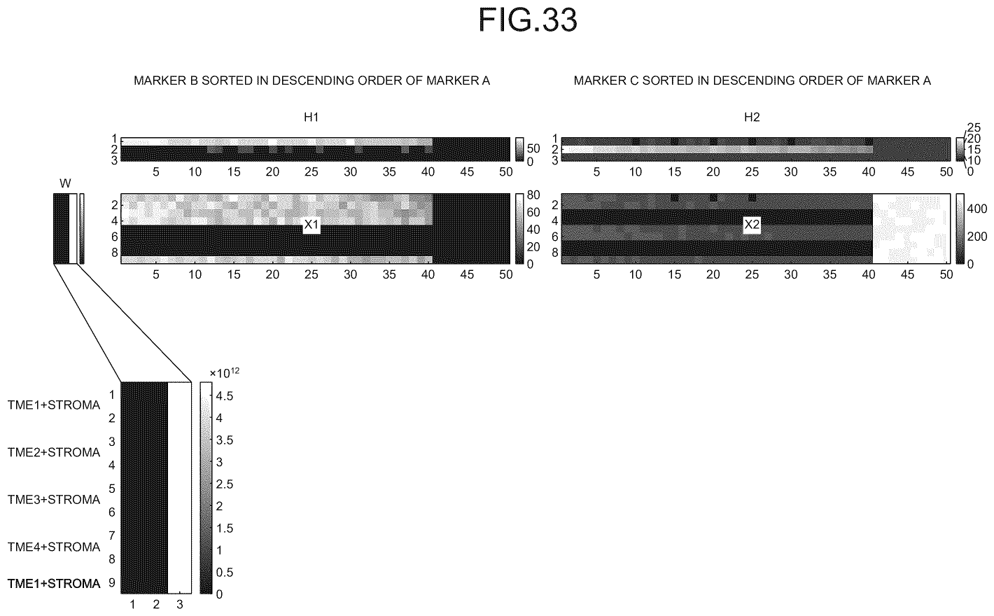

The third calculation method according to the present embodiment will be described with reference to FIGS. 32 and 33. FIG. 32 is a diagram for describing the third calculation method according to the present embodiment. FIG. 33 is a diagram for describing a result of the JNMF using an initial value calculated by the third calculation method according to the present embodiment.

As illustrated in FIG. 32, the correlation analysis unit 133d performs LSM with X and H (X1 and H1 of the marker B and X2 and H2 of the marker C) for each marker to acquire W for each marker, and employs an acquired average value of W (W1, W2) for each marker as a predetermined initial value. For example, the correlation analysis unit 133d uses JNMF calculation results of H1 and H2 of only the data set of the existing samples 1 to 8, and employs an average value of the initial value of X1/H1=W1 and the initial value of X2/H2=W2 as the predetermined initial value.

Specifically, the correlation analysis unit 133d performs LSM with X1 including the new sample 9 and H1 calculated by executing the JNMF with only the data set of the existing samples 1 to 8, and calculates the value of W1 including the new sample 9. In addition, the correlation analysis unit 133d performs LSM with X2 including the new sample 9 and H1 calculated by executing the JNMF with only the data set of the existing samples 1 to 8, and calculates the value of W2 including the new sample 9. The correlation analysis unit 133d calculates an average value of the values of W1 and W2, and employs the calculated average value as a predetermined initial value. The average value of the value of W1 and the value of W2 includes the value of xyz of the new sample 9.

Thereafter, the correlation analysis unit 133d gives the calculated initial value to the JNMF, and performs the JNMF on the simulation data set obtained by adding the new sample 9 to the existing samples 1 to 8. Thus, as illustrated in FIG. 33, the samples 1 to 9 is divided into clusters. In the present simulation data, the new sample 9 (TME1+stromal cells) was not divided into clusters as expected. However, it is known that new samples are divided into clusters as expected depending on other simulation data.

### (Fourth Calculation Method)

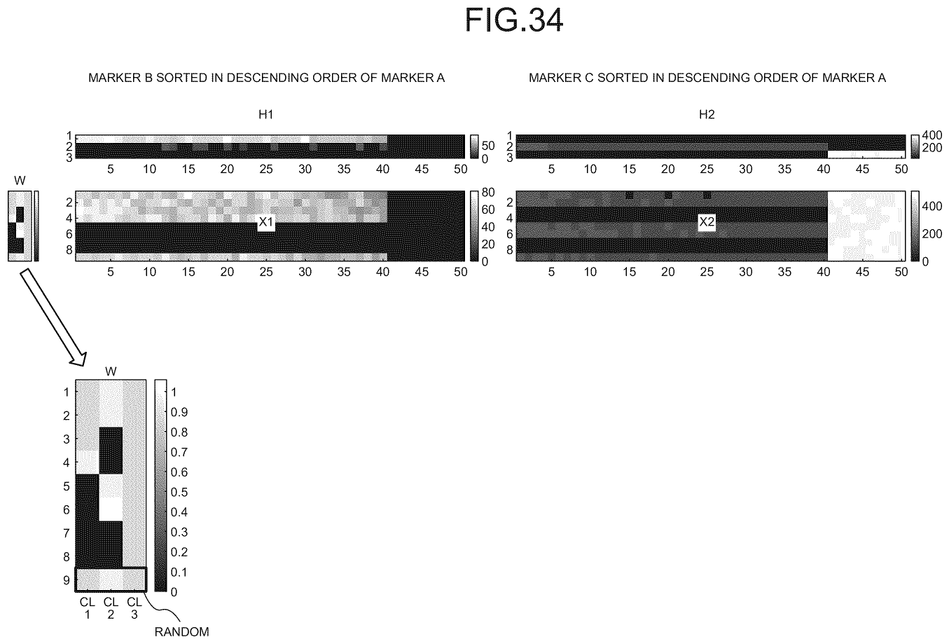

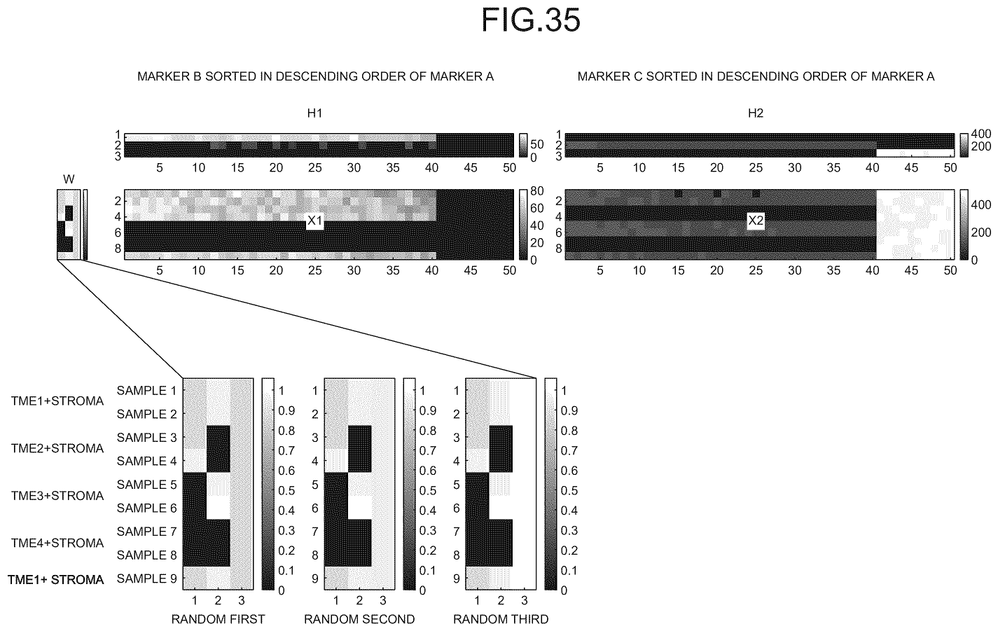

The fourth calculation method according to the present embodiment will be described with reference to FIGS. 34 and 35. FIG. 34 is a diagram for describing the fourth calculation method according to the present embodiment. FIG. 35 is a diagram for describing a result of the JNMF using an initial value calculated by the fourth calculation method according to the present embodiment.

As illustrated in FIG. 34, the correlation analysis unit 133d employs a random number as a predetermined initial value only for the added new sample. For example, the correlation analysis unit 133d uses a binarized value as a predetermined initial value for the portion of the data set of the existing samples 1 to 8, and uses a random number between 0 and 1 only for the new sample 9 as a predetermined initial value (xyz value of the new sample 9). That is, the predetermined initial value of the new sample 9 is random.

Thereafter, the correlation analysis unit 133d gives the calculated initial value to the JNMF, and performs the JNMF on the simulation data set obtained by adding the new sample 9 to the existing samples 1 to 8. Thus, as illustrated in FIG. 35, the samples 1 to 9 are divided into clusters for each execution count. In all of random first, random second and random third, the new sample 9 (TME1+stromal cells) was divided into clusters as expected. Note that, in the example of FIG. 35, the number of executions is 3, but the initial value of W is between 0 to 1.

### (Fifth Calculation Method)

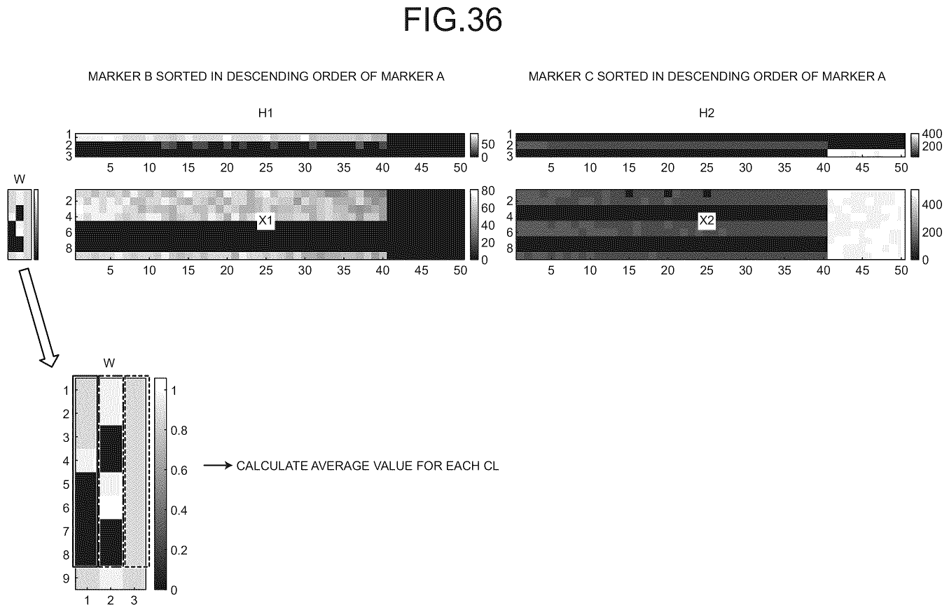

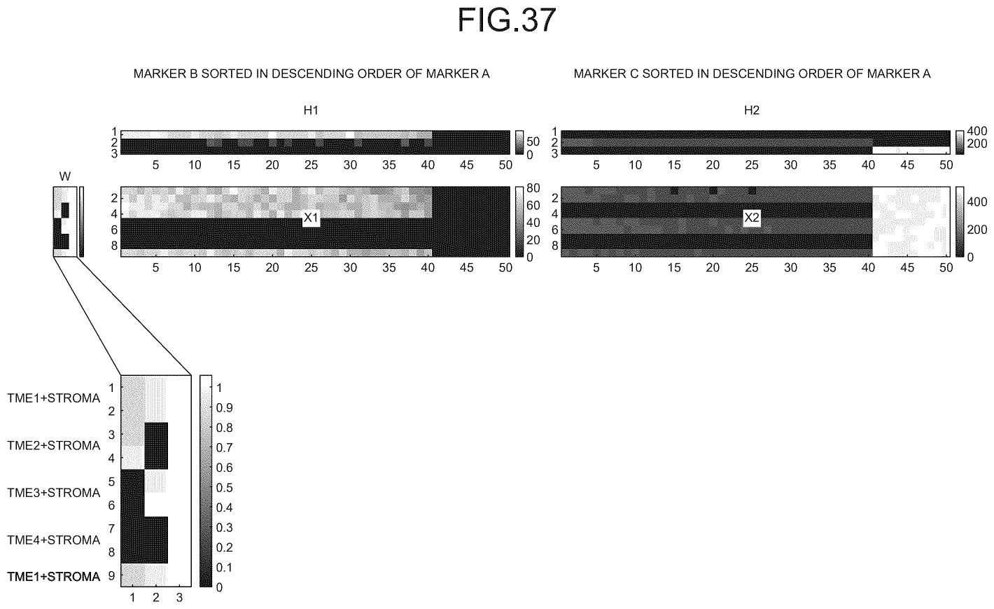

The fifth calculation method according to the present embodiment will be described with reference to FIGS. 36 and 37. FIG. 36 is a diagram for describing the fifth calculation method according to the present embodiment. FIG. 37 is a diagram for describing a result of the JNMF using an initial value calculated by the fifth calculation method according to the present embodiment.

As illustrated in FIG. 36, the correlation analysis unit 133d employs an average value of W for each cluster as a predetermined initial value. For example, the correlation analysis unit 133d uses W that is a result of executing the JNMF on a data set of the existing samples 1 to 8, and uses an average value of W for each cluster as an initial value of the new sample 9 (a value of xyz of the new sample 9).

Thereafter, the correlation analysis unit 133d gives the calculated initial value to the JNMF, and performs the JNMF on the simulation data set obtained by adding the new sample 9 to the existing samples 1 to 8. Thus, as illustrated in FIG. 37, the samples 1 to 9 are divided into clusters. The new sample 9 (TME1+stromal cells) segmented into clusters as expected.

### (Only Clustering)

Note that, in a case where it is desired to add a new sample and perform only clustering, as in the first calculation method, LSM is performed with a matrix obtained by connecting H1, H2, . . . , and Hn for which the JNMF is performed on the data set of the existing samples 1 to 8 and a matrix obtained by connecting X1, X2, . . . , and Xn including the new sample 9, and clustering can be performed on the basis of the calculated value of W.

### (Utilization of Data)

When the values of results (H and W) obtained by executing the JNMF with the original matrices X1, X2, . . . , and Xn are disclosed, clustering can be performed by executing the JNMF and feature regions can be extracted even in an institution having an insufficient number of samples or an institution having no reference data. Further, it is also possible to compare features with samples of other periods. In addition, in the case of only clustering, data can be shared only by numerical values without using a whole slide imaging (WSI) image, and thus it is possible to implement compression of a data size.

### <2-4. Operation and Effect>

As described above, according to the present embodiment, effects similar to those of the first embodiment can be obtained. Furthermore, according to the present embodiment, the correlation analysis unit 133d gives a predetermined initial value to the JNMF. Thus, the result of the JNMF is stabilized, and hence the correlation between the plurality of biomarkers can be accurately obtained.

In addition, the correlation analysis unit 133d may connect the matrix X and the matrix H for each biomarker, perform the least squares method on the matrix X and the matrix H for each biomarker connected to acquire the matrix W, and employ the acquired matrix Was a predetermined initial value. Thus, the result of the JNMF can be stabilized.

In addition, the correlation analysis unit 133d may perform the least squares method on the matrix X and the matrix H for each biomarker to acquire the matrix W for each biomarker, and may employ the matrix W having the smallest residual from the acquired matrix W for each biomarker as a predetermined initial value. Thus, the result of the JNMF can be stabilized.

In addition, the correlation analysis unit 133d may perform the least squares method on the matrix X and the matrix H for each biomarker to acquire the matrix W for each biomarker, and employ an average value of the acquired matrices W for each biomarker as a predetermined initial value. Thus, the result of the JNMF can be stabilized.

In addition, the predetermined initial value (for example, an initial value of W) may include an initial value (for example, the value of xyz of W of the new sample 9) related to the new sample to be added, and the correlation analysis unit 133d may employ a random number only for the initial value related to the new sample. Thus, the result of the JNMF can be stabilized.

In addition, the predetermined initial value (for example, an initial value of W) may include an initial value (for example, the value of xyz of W of the new sample 9) related to the new sample to be added, and the correlation analysis unit 133d may employ an average value of W for each of clusters of the clustering processing as the initial value related to the new sample. Thus, the result of the JNMF can be stabilized.

### 3. Other Embodiments

The processing according to the above-described embodiment (examples and modifications) may be performed in various different modes other than the above-described embodiment. For example, among the processes described in the above embodiments, all or part of the processes described as being performed automatically can be performed manually, or all or part of the processes described as being performed manually can be performed automatically by a publicly known method. Further, the processing procedure, specific name, and information including various data and parameters depicted in the above document and the drawings can be arbitrarily changed unless otherwise specified. For example, the various types of information depicted in each figure are not limited to the depicted information.

Further, each component of each device depicted in the drawings is functionally conceptual, and is not necessarily physically configured as depicted in the drawings. That is, a specific form of distribution and integration of each device is not limited to the depicted form, and all or a part thereof can be functionally or physically distributed and integrated in any unit according to various loads, usage conditions, and the like.

In addition, the above-described embodiments can be appropriately combined within a range in which the processing contents do not contradict each other. Further, the effects described in the present description are merely examples and are not limited, and other effects may be provided.

### 4. Application Example

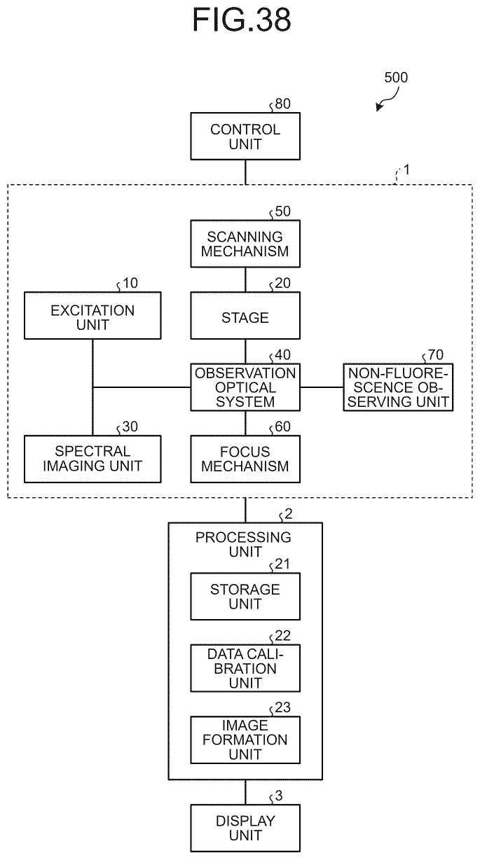

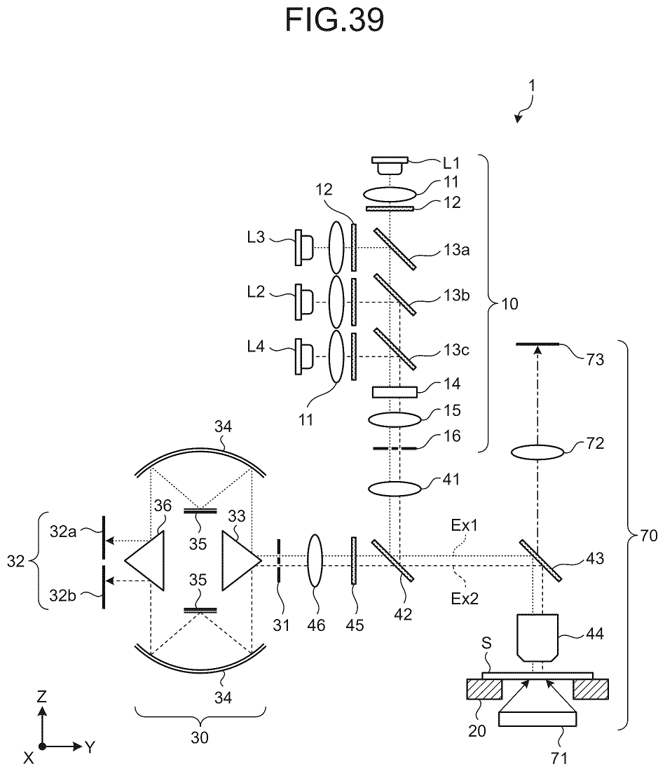

The technology according to the present disclosure can be applied to, for example, a fluorescence observation apparatus 500 (an example of a microscope system) or the like. Hereinafter, a configuration example of an applicable fluorescence observation apparatus 500 will be described with reference to FIGS. 38 and 39. FIG. 38 is a diagram showing an example of a schematic configuration of the fluorescence observation apparatus 500 according to the present embodiment. FIG. 39 is a diagram showing an example of a schematic configuration of an observation unit 1 according to the present embodiment.

As shown in FIG. 38, the fluorescence observation apparatus 500 includes the observation unit 1, a process unit 2, and a display unit 3.

The observation unit 1 includes an excitation unit (irradiation unit) 10, a stage 20, a spectral imaging unit 30, an observation optical system 40, a scanning mechanism 50, a focus mechanism 60, and a non-fluorescence observing unit 70.

The excitation unit 10 irradiates the observation target with a plurality of beams of irradiation light having different wavelengths. For example, the excitation unit 10 irradiates a pathological specimen (pathological sample), which is the observation target, with a plurality of line illuminations having different wavelengths arranged in parallel with different axes. The stage 20 is a table that supports the pathological specimen, and is configured to be movable in a direction perpendicular to the direction of line light by the line illuminations by the scanning mechanism 50. The spectral imaging unit 30 includes a spectroscope and acquires a fluorescence spectrum (spectroscopic data) of the pathological specimen excited linearly by the line illuminations.

That is, the observation unit 1 functions as a line spectroscope that acquires spectroscopic data corresponding to the line illuminations. Further, the observation unit 1 also functions as an imaging device that captures a plurality of fluorescence images generated by an imaging target (pathological specimen) for each of a plurality of fluorescence wavelengths for each line and acquires data of the plurality of captured fluorescence images in an arrangement order of the lines.

Here, parallel with different axis means that the plurality of line illuminations has different axes and are parallel. The different axes mean that the axes are not coaxial, and the distance between the axes is not particularly limited. The parallel is not limited to parallel in a strict sense, and includes a state of being substantially parallel. For example, there may be distortion originated from an optical system such as a lens or deviation from a parallel state due to manufacturing tolerance, and this case is also regarded as parallel.

The excitation unit 10 and the spectral imaging unit 30 are connected to the stage 20 via the observation optical system 40. The observation optical system 40 has a function of following an optimum focus by the focus mechanism 60. The non-fluorescence observing unit 70 for performing dark field observation, bright field observation, and the like may be connected to the observation optical system 40. In addition, a control unit 80 that controls the excitation unit 10, the spectral imaging unit 30, the scanning mechanism 50, the focus mechanism 60, the non-fluorescence observing unit 70, and the like may be connected to the observation unit 1.

The process unit 2 includes a storing unit 21, a data calibration unit 22, and an image formation unit 23. The process unit 2 typically forms an image of the pathological specimen or outputs a distribution of the fluorescence spectrum on the basis of the fluorescence spectrum of the pathological specimen (hereinafter also referred to as a sample S) acquired by the observation unit 1. The image referred to herein refers to a constituent ratio of autofluorescence derived from a dye or a sample, or the like constituting the spectrum, an image converted from waveforms into RGB (red, green, and blue) color, a luminance distribution in a specific wavelength band, and the like.

The storing unit 21 includes a nonvolatile storage medium such as a hard disk drive or a flash memory, and a storage control unit that controls writing and reading of data to and from the storage medium. The storing unit 21 stores spectroscopic data indicating a correlation between each wavelength of light emitted by each of the plurality of line illuminations included in the excitation unit 10 and fluorescence received by the camera of the spectral imaging unit 30. Further, the storing unit 21 stores in advance information indicating a standard spectrum of autofluorescence related to a sample (pathological specimen) to be observed and information indicating a standard spectrum of a single dye staining the sample.

The data calibration unit 22 configures the spectroscopic data stored in the storing unit 21 on the basis of the captured image captured by the camera of the spectral imaging unit 30. The image formation unit 23 forms a fluorescence image of the sample on the basis of the spectroscopic data and an interval Δy of the plurality of line illuminations irradiated by the excitation unit 10. For example, the process unit 2 including the data calibration unit 22, the image formation unit 23, and the like is implemented by hardware elements used in a computer such as a central processing unit (CPU), a random access memory (RAM), and a read only memory (ROM), and a necessary program (software). Instead of or in addition to the CPU, a programmable logic device (PLD) such as a field programmable gate array (FPGA), a digital signal processor (DSP), an application specific integrated circuit (ASIC), or the like may be used.

The display unit 3 displays, for example, various types of information such as an image based on the fluorescence image formed by the image formation unit 23. The display unit 3 may include, for example, a monitor integrally attached to the process unit 2, or may be a display device connected to the process unit 2. The display unit 3 includes, for example, a display element such as a liquid crystal device or an organic EL device, and a touch sensor, and is configured as a user interface (UI) that displays input settings of image-capturing conditions, a captured image, and the like.

Next, details of the observation unit 1 will be described with reference to FIG. 39. Here, a description will be given on the assumption that the excitation unit 10 includes two line illuminations Ex1 and Ex2 that each emit light of two wavelengths. For example, the line illumination Ex1 emits light having a wavelength of 405 nm and light having a wavelength of 561 nm, and the line illumination Ex2 emits light having a wavelength of 488 nm and light having a wavelength of 645 nm.

As shown in FIG. 39, the excitation unit 10 includes a plurality of excitation light sources L1, L2, L3, and L4 (four excitation light sources in this example). Each of the excitation light sources L1 to L4 includes a laser light source that outputs laser light having a wavelength of 405 nm, 488 nm, 561 nm, and 645 nm, respectively. For example, each of the excitation light sources L1 to L4 includes a light emitting diode (LED), a laser diode (LD), or the like.

Furthermore, the excitation unit 10 includes a plurality of collimator lenses 11, a plurality of laser line filters 12, a plurality of dichroic mirrors 13a, 13b, and 13c, a homogenizer 14, a condenser lens 15, and an incident slit 16 so as to correspond to each of the excitation light sources L1 to L4.

The laser light emitted from the excitation light source L1 and the laser light emitted from the excitation light source L3 are collimated by the collimator lens 11, transmitted through the laser line filter 12 for cutting a skirt of each wavelength band, and made coaxial by the dichroic mirror 13a. The two coaxial laser lights are further beam-shaped by the homogenizer 14 such as a fly-eye lens and the condenser lens 15 so as to be the line illumination Ex1.

Similarly, the laser light emitted from the pumping light source L2 and the laser light emitted from the excitation light source L4 are coaxial by the dichroic mirrors 13b and 13c, and line illumination is performed so that the line illumination Ex2 is different in axis from the line illumination Ex1. The line illuminations Ex1 and Ex2 form line illuminations with different axes (primary image), which are separated by a distance Δy in the incident slit 16 (slit conjugate) having a plurality of slit portions through which each of the line illuminations Ex1 and Ex2 can pass.

Note that, in the present embodiment, an example in which the four lasers have two coaxial axes and two different axes will be described, but in addition to this, the two lasers may have two different axes or the four lasers may have four different axes.

The sample S on the stage 20 is irradiated with the primary image via the observation optical system 40. The observation optical system 40 includes a condenser lens 41, dichroic mirrors 42 and 43, an objective lens 44, a band pass filter 45, and a condenser lens (an example of an imaging lens) 46. The line illuminations Ex1 and Ex2 are collimated by the condenser lens 41 paired with the objective lens 44, reflected by the dichroic mirrors 42 and 43, transmitted through the objective lens 44, and irradiates the sample S on the stage 20.

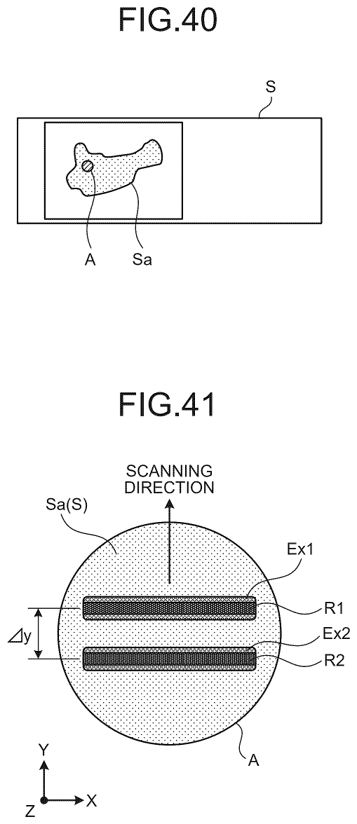

Here, FIG. 40 is a diagram showing an example of the sample S according to the present embodiment. FIG. 40 shows a state in which the sample S is viewed from the irradiation directions of the line illuminations Ex1 and Ex2 as excitation light. The sample S is typically configured by a slide including an observation target Sa such as a tissue section as shown in FIG. 40, but may be of course other than that. The observation target Sa is, for example, a biological sample such as a nucleic acid, a cell, a protein, a bacterium, or a virus. The sample S (observation target Sa) is stained with a plurality of fluorescent dyes. The observation unit 1 enlarges and observes the sample S at a desired magnification.

FIG. 41 is an enlarged diagram showing a region A in which the sample S according to the present embodiment is irradiated with the line illuminations Ex1 and Ex2. In the example of FIG. 41, two line illuminations Ex1 and Ex2 are arranged in the region A, and imaging areas R1 and R2 of the spectral imaging unit 30 are arranged so as to overlap the line illuminations Ex1 and Ex2. The two line illuminations Ex1 and Ex2 are each parallel to a Z-axis direction and are arranged apart from each other by a predetermined distance Δy in a Y-axis direction.

The line illuminations Ex1 and Ex2 are formed on the surface of the sample S as shown in FIG. 41. As shown in FIG. 39, fluorescence excited in the sample S by the line illuminations Ex1 and Ex2 is condensed by the objective lens 44, reflected by the dichroic mirror 43, transmitted through the dichroic mirror 42 and the band pass filter 45 that cuts off the excitation light, condensed again by the condenser lens 46, and incident on the spectral imaging unit 30.

As shown in FIG. 39, the spectral imaging unit 30 includes an observation slit (opening) 31, an imaging element 32, a first prism 33, a mirror 34, a diffraction grating 35 (wavelength dispersion element), and a second prism 36.

In the example of FIG. 39, the imaging element 32 includes two imaging elements 32a and 32b. The imaging element 32 captures (receives) a plurality of light beams (fluorescence and the like) wavelength-dispersed by the diffraction grating 35. As the imaging element 32, for example, a two-dimensional imager such as a charge coupled device (CCD) or a complementary metal oxide semiconductor (CMOS) is employed.

The observation slit 31 is disposed at the condensing point of the condenser lens 46, and has the same number of (two this example) slit portions as the number of excitation lines. The fluorescence spectra derived from the two excitation lines that have passed through the observation slit 31 are separated by the first prism 33 and reflected by a grating surface of the diffraction grating 35 via the mirror 34, so that the fluorescence spectra are further separated into fluorescence spectra of respective excitation wavelengths. The four separated fluorescence spectra are incident on the imaging elements 32a and 32b via the mirror 34 and the second prism 36, and are developed as spectroscopic data into spectroscopic data (x, λ) expressed by the position x in the line direction and the wavelength λ. The spectroscopic data (x, λ) is a pixel value of a pixel at a position x in a row direction and at a position of a wavelength λ in a column direction among pixels included in the imaging element 32. Note that the spectroscopic data (x, λ) may be simply described as spectroscopic data.

Note that the pixel size (nm/Pixel) of the imaging elements 32a and 32b is not particularly limited, and is set, for example, equal to or more than 2 (nm/Pixel) and equal to or less than 20 (nm/Pixel). This dispersion value may be achieved optically or at a pitch of the diffraction grating 35, or may be achieved by using hardware binning of the imaging elements 32a and 32b. In addition, the dichroic mirror 42 and the band pass filter 45 are inserted in the middle of the optical path so that the excitation light (line illuminations Ex1 and Ex2) does not reach the imaging element 32.

Each of the line illuminations Ex1 and Ex2 is not limited to the case of being configured with a single wavelength, and each may be configured with a plurality of wavelengths. When the line illuminations Ex1 and Ex2 are each formed by a plurality of wavelengths, the fluorescence excited by these also includes a plurality of spectra. In this case, the spectral imaging unit 30 includes a wavelength dispersion element for separating the fluorescence into a spectrum derived from the excitation wavelength. The wavelength dispersion element includes a diffraction grating, a prism, or the like, and is typically disposed on an optical path between the observation slit 31 and the imaging element 32.

Note that the stage 20 and the scanning mechanism 50 constitute an X-Y stage, and move the sample S in the X-axis direction and the Y-axis direction in order to acquire a fluorescence image of the sample S. In the whole slide imaging (WSI), an operation of scanning the sample S in the Y-axis direction, then moving the sample S in the X-axis direction, and further performing scanning in the Y-axis direction is repeated. By using the scanning mechanism 50, it is possible to continuously acquire dye spectra (fluorescence spectra) excited at different excitation wavelengths, which are spatially separated by the distance Δy on the sample S (observation target Sa) in the Y-axis direction.

The scanning mechanism 50 changes the position irradiated with the irradiation light in the sample S over time. For example, the scanning mechanism 50 scans the stage 20 in the Y-axis direction. The scanning mechanism 50 can cause the stage 20 to scan the plurality of line illuminations Ex1 and Ex2 in the Y-axis direction, that is, in the arrangement direction of the line illuminations Ex1 and Ex2. This is not limited to this example, and the plurality of line illuminations Ex1 and Ex2 may be scanned in the Y-axis direction by a galvano mirror disposed in the middle of the optical system. Since the data derived from each of the line illuminations Ex1 and Ex2 (for example, the two-dimensional data or the three-dimensional data) is data whose coordinates are shifted by the distance Δy with respect to the Y axis, the data is corrected and output on the basis of the distance Δy stored in advance or the value of the distance Δy calculated from the output of the imaging element 32.

As shown in FIG. 39, the non-fluorescence observing unit 70 includes a light source 71, the dichroic mirror 43, the objective lens 44, a condenser lens 72, an imaging element 73, and the like. In the non-fluorescence observing unit 70, an observation system by dark field illumination is shown in the example of FIG. 39.

The light source 71 is disposed on the side facing the objective lens 44 with respect to the stage 20, and irradiates the sample S on the stage 20 with illumination light from the side opposite to the line illuminations Ex1 and Ex2. In a case of the dark field illumination, the light source 71 illuminates from the outside of the NA (numerical aperture) of the objective lens 44, and light (dark field image) diffracted by the sample S is imaged by the imaging element 73 via the objective lens 44, the dichroic mirror 43, and the condenser lens 72. By using dark field illumination, even a apparently transparent sample such as a fluorescently-stained sample can be observed with contrast.

Note that this dark field image may be observed simultaneously with fluorescence and used for real-time focusing. In this case, as the illumination wavelength, a wavelength that does not affect fluorescence observation may be selected. The non-fluorescence observing unit 70 is not limited to the observation system that acquires a dark field image, and may be configured by an observation system that can acquire a non-fluorescence image such as a bright field image, a phase difference image, a phase image, and an in-line hologram image. For example, as a method for acquiring a non-fluorescence image, various observation methods such as a Schlieren method, a phase difference contrast method, a polarization observation method, and an epi-illumination method can be employed. The position of the illumination light source is not limited to below the stage 20, and may be above the stage 20 or around the objective lens 44. In addition, not only a method of performing focus control in real time, but also another method such as a prefocus map method of recording focus coordinates (Z coordinates) in advance may be employed.

Note that, in the above description, the line illumination as the excitation light includes two line illuminations Ex1 and Ex2 but is not limited thereto, and may be three, four, or five or more. In addition, each line illumination may include a plurality of excitation wavelengths selected so that the color separation performance is not degraded as much as possible. Further, even if there is one line illumination, if it is an excitation light source including a plurality of excitation wavelengths and each excitation wavelength is recorded in association with the data acquired by the imaging element 32, it is possible to obtain a polychromatic spectrum although it is not possible to obtain separability to be parallel to different axes.

The application example in which the technology according to the present disclosure is applied to the fluorescence observation apparatus 500 has been described above. Note that the above-described configuration described with reference to FIGS. 38 and 39 is merely an example, and the configuration of the fluorescence observation apparatus 500 according to the present embodiment is not limited to such an example. For example, the fluorescence observation apparatus 500 may not necessarily include all of the configurations shown in FIGS. 38 and 39, and may include a configuration not shown in FIGS. 38 and 39.

### 5. Application Example

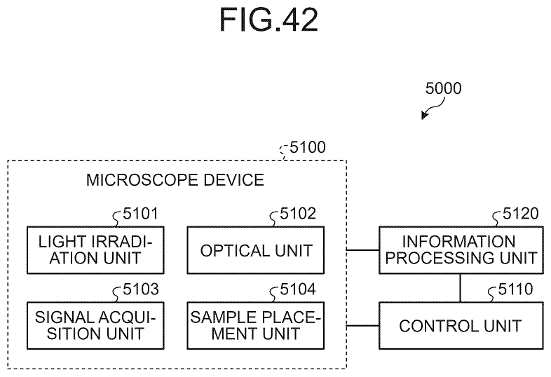

The technology according to the present disclosure can be applied to, for example, a microscope system and the like. Hereinafter, a configuration example of a microscope system 5000 that can be applied will be described with reference to FIGS. 42 to 44. A microscope device 5100 which is a part of the microscope system 5000 functions as an imaging device.

FIG. 42 shows an example configuration of a microscope system of the present disclosure. A microscope system 5000 shown in FIG. 42 includes a microscope device 5100, a control unit 5110, and an information processing unit 5120. The microscope device 5100 includes a light irradiation unit 5101, an optical unit 5102, and a signal acquisition unit 5103. The microscope device 5100 may further include a sample placement unit 5104 on which a biological sample S is placed. Note that the configuration of the microscope device 5100 is not limited to that shown in FIG. 42. For example, the light irradiation unit 5101 may exist outside the microscope device 5100, and a light source not included in the microscope device 5100 may be used as the light irradiation unit 5101. Alternatively, the light irradiation unit 5101 may be disposed so that the sample placement unit 5104 is sandwiched between the light irradiation unit 5101 and the optical unit 5102, and may be disposed on the side at which the optical unit 5102 exists, for example. The microscope device 5100 may be designed to be capable of performing one or more of the following: bright-field observation, phase contrast observation, differential interference contrast observation, polarization observation, fluorescent observation, and darkfield observation.

The microscope system 5000 may be designed as a so-called whole slide imaging (WSI) system or a digital pathology imaging system, and can be used for pathological diagnosis. Alternatively, the microscope system 5000 may be designed as a fluorescence imaging system, or particularly, as a multiple fluorescence imaging system.

For example, the microscope system 5000 may be used to make an intraoperative pathological diagnosis or a telepathological diagnosis. In the intraoperative pathological diagnosis, the microscope device 5100 can acquire the data of the biological sample S acquired from the subject of the operation while the operation is being performed, and then transmit the data to the information processing unit 5120. In the telepathological diagnosis, the microscope device 5100 can transmit the acquired data of the biological sample S to the information processing unit 5120 located in a place away from the microscope device 5100 (such as in another room or building). In these diagnoses, the information processing unit 5120 then receives and outputs the data. On the basis of the output data, the user of the information processing unit 5120 can make a pathological diagnosis.

### (Biological Sample)

The biological sample S may be a sample containing a biological component. The biological component may be a tissue, a cell, a liquid component of the living body (blood, urine, or the like), a culture, or a living cell (a myocardial cell, a nerve cell, a fertilized egg, or the like). The biological sample may be a solid, or may be a specimen fixed with a fixing reagent such as paraffin or a solid formed by freezing. The biological sample can be a section of the solid. A specific example of the biological sample may be a section of a biopsy sample.

The biological sample may be one that has been subjected to a treatment such as staining or labeling. The treatment may be staining for indicating the morphology of the biological component or for indicating the substance (surface antigen or the like) contained in the biological component, and can be hematoxylin-eosin (HE) staining or immunohistochemistry staining, for example. The biological sample may be one that has been subjected to the above treatment with one or more reagents, and the reagent(s) can be a fluorescent dye, a coloring reagent, a fluorescent protein, or a fluorescence-labeled antibody.

The specimen may be prepared from a tissue sample for the purpose of pathological diagnosis or clinical examination. Alternatively, the specimen is not necessarily of the human body, and may be derived from an animal, a plant, or some other material. The specimen may differ in property, depending on the type of the tissue being used (such as an organ or a cell, for example), the type of the disease being examined, the attributes of the subject (such as age, gender, blood type, and race, for example), or the subject's daily habits (such as an eating habit, an exercise habit, and a smoking habit, for example). The specimen may be accompanied by identification information (bar code, QR code (registered trademark), or the like) for identifying each specimen, and be managed in accordance with the identification information.

### (Light Irradiation Unit)

The light irradiation unit 5101 is a light source for illuminating the biological sample S, and is an optical unit that guides light emitted from the light source to a specimen. The light source can illuminate a biological sample with visible light, ultraviolet light, infrared light, or a combination thereof. The light source may be one or more of the following: a halogen light source, a laser light source, an LED light source, a mercury light source, and a xenon light source. The light source in fluorescent observation may be of a plurality of types and/or wavelengths, and the types and the wavelengths may be appropriately selected by a person skilled in the art. The light irradiation 5101 unit may have a configuration of a transmissive type, a reflective type, or an epi-illumination type (a coaxial epi-illumination type or a side-illumination type).

### (Optical Unit)

The optical unit 5102 is designed to guide the light from the biological sample S to the signal acquisition unit 5103. The optical unit 5102 may be designed to enable the microscope device 5100 to observe or capture an image of the biological sample S. The optical unit 5102 may include an objective lens. The type of the objective lens may be appropriately selected by a person skilled in the art, in accordance with the observation method. The optical unit 5102 may also include a relay lens for relaying an image magnified by the objective lens to the signal acquisition unit 5103. The optical unit 5102 may further include optical components other than the objective lens and the relay lens, and the optical components may be an eyepiece, a phase plate, a condenser lens, and the like. The optical unit 5102 may further include a wavelength separation unit designed to separate light having a predetermined wavelength from the light from the biological sample S. The wavelength separation unit may be designed to selectively cause light having a predetermined wavelength or a predetermined wavelength range to reach the signal acquisition unit 5103. The wavelength separation unit may include one or more of the following: a filter, a polarizing plate, a prism (Wollaston prism), and a diffraction grating that selectively pass light, for example. The optical component(s) included in the wavelength separation unit may be disposed in the optical path from the objective lens to the signal acquisition unit 5103, for example. The wavelength separation unit is provided in the microscope device 5100 in a case where fluorescent observation is performed, or particularly, where an excitation light irradiation unit is included. The wavelength separation unit may be designed to separate fluorescence or white light from fluorescence.

### (Signal Acquisition Unit)

The signal acquisition unit 5103 may be designed to receive light from the biological sample S, and convert the light into an electrical signal, or particularly, into a digital electrical signal. The signal acquisition unit 5103 may be designed to be capable of acquiring data about the biological sample S, on the basis of the electrical signal. The signal acquisition unit 5103 may be designed to be capable of acquiring data of an image (a captured image, or particularly, a still image, a time-lapse image, or a moving image) of the biological sample S, or particularly, may be designed to acquire data of an image enlarged by the optical unit 5102. The signal acquisition unit 5103 includes one or more image sensors, CMOSs, CCDs, or the like that include a plurality of pixels arranged in one- or two-dimensional manner. The signal acquisition unit 5103 may include an image sensor for acquiring a low-resolution image and an image sensor for acquiring a high-resolution image, or may include an image sensor for sensing for AF or the like and an image sensor for outputting an image for observation or the like. The image sensor may include not only the plurality of pixels, but also a signal processing unit (including one or more of the following: a CPU, a DSP, and a memory) that performs signal processing using pixel signals from the respective pixels, and an output control unit that controls outputting of image data generated from the pixel signals and processed data generated by the signal processing unit. The image sensor including the plurality of pixels, the signal processing unit, and the output control unit can be preferably designed as a one-chip semiconductor device. Note that the microscope system 5000 may further include an event detection sensor. The event detection sensor includes a pixel that photoelectrically converts incident light, and may be designed to detect that a change in the luminance of the pixel exceeds a predetermined threshold, and regard the change as an event. The event detection sensor may be of an asynchronous type.

### (Control Unit)

The control unit 5110 controls imaging being performed by the microscope device 5100. For the imaging control, the control unit 5110 can drive movement of the optical unit 5102 and/or the sample placement unit 5104, to adjust the positional relationship between the optical unit 5102 and the sample placement unit 5104. The control unit 5110 can move the optical unit 5102 and/or the sample placement unit 5104 in a direction toward or away from each other (in the optical axis direction of the objective lens, for example). The control unit 5110 may also move the optical unit 5102 and/or the sample placement unit 5104 in any direction in a plane perpendicular to the optical axis direction. For the imaging control, the control unit 5110 may control the light irradiation unit 5101 and/or the signal acquisition unit 5103.

### (Sample Placement Unit)

The sample placement unit 5104 may be designed to be capable of securing the position of a biological sample on the sample placement unit 5104, and may be a so-called stage. The sample placement unit 5104 may be designed to be capable of moving the position of the biological sample in the optical axis direction of the objective lens and/or in a direction perpendicular to the optical axis direction.

### (Information Processing Unit)

The information processing unit 5120 can acquire, from the microscope device 5100, data (imaging data or the like) acquired by the microscope device 5100. The information processing unit 5120 can perform image processing on the imaging data. The image processing may include an unmixing process, or more specifically, a spectral unmixing process. The unmixing process may include a process of extracting data of the optical component of a predetermined wavelength or in a predetermined wavelength range from the imaging data to generate image data, or a process of removing data of the optical component of a predetermined wavelength or in a predetermined wavelength range from the imaging data. The image processing may also include an autofluorescence separation process for separating the autofluorescence component and the dye component of a tissue section, and a fluorescence separation process for separating wavelengths between dyes having different fluorescence wavelengths from each other. The autofluorescence separation process may include a process of removing the autofluorescence component from image information about another specimen, using an autofluorescence signal extracted from one specimen of the plurality of specimens having the same or similar properties. The information processing unit 5120 may transmit data for the imaging control to the control unit 5110, and the control unit 5110 that has received the data may control the imaging being by the microscope device 5100 in accordance with the data.

The information processing unit 5120 may be designed as an information processing device such as a general-purpose computer, and may include a CPU, RAM, and ROM. The information processing unit 5120 may be included in the housing of the microscope device 5100, or may be located outside the housing. Further, the various processes or functions to be executed by the information processing unit 5120 may be realized by a server computer or a cloud connected via a network.

The method to be implemented by the microscope device 5100 to capture an image of the biological sample S may be appropriately selected by a person skilled in the art, in accordance with the type of the biological sample, the purpose of imaging, and the like. Examples of the imaging method are described below.

One example of the imaging method is as follows. The microscope device 5100 can first identify an imaging target region. The imaging target region may be identified so as to cover the entire region in which the biological sample exists, or may be identified so as to cover the target portion (the portion in which the target tissue section, the target cell, or the target lesion exists) of the biological sample. Next, the microscope device 5100 divides the imaging target region into a plurality of divided regions of a predetermined size, and the microscope device 5100 sequentially captures images of the respective divided regions. As a result, an image of each divided region is acquired.

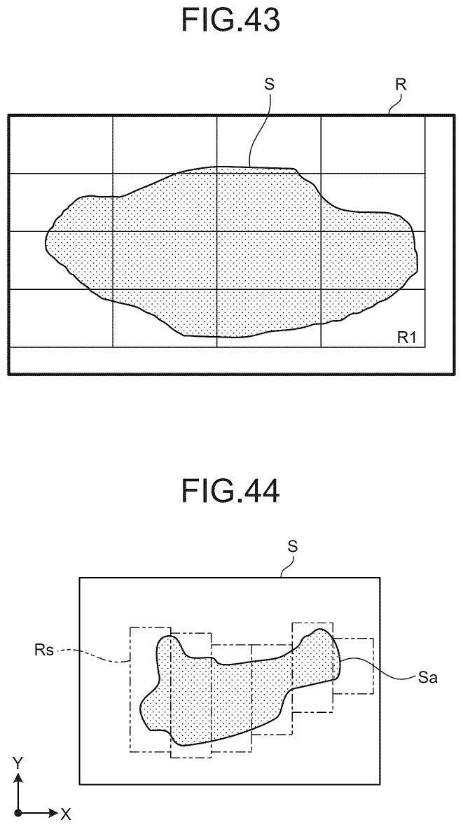

As shown in FIG. 43, the microscope device 5100 identifies an imaging target region R that covers the entire biological sample S. The microscope device 5100 then divides the imaging target region R into 16 divided regions. The microscope device 5100 then captures an image of a divided region R1, and next captures one of the regions included in the imaging target region R, such as an image of a region adjacent to the divided region R1. After that, divided region imaging is performed until images of all the divided regions have been captured. Note that an image of a region other than the imaging target region R may also be captured on the basis of captured image information about the divided regions. The positional relationship between the microscope device 5100 and the sample placement unit 5104 is adjusted so that an image of the next divided region is captured after one divided region is captured. The adjustment may be performed by moving the microscope device 5100, moving the sample placement unit 5104, or moving both. In this example, the imaging device that captures an image of each divided region may be a two-dimensional image sensor (an area sensor) or a one-dimensional image sensor (a line sensor). The signal acquisition unit 5103 may capture an image of each divided region via the optical unit 5102. Further, images of the respective divided regions may be continuously captured while the microscope device 5100 and/or the sample placement unit 5104 is moved, or movement of the microscope device 5100 and/or the sample placement unit 5104 may be stopped every time an image of a divided region is captured. The imaging target region may be divided so that the respective divided regions partially overlap, or the imaging target region may be divided so that the respective divided regions do not overlap. A plurality of images of each divided region may be captured while the imaging conditions such as the focal length and/or the exposure time are changed. The information processing device can also generate image data of a wider region by stitching a plurality of adjacent divided regions. As the stitching process is performed on the entire imaging target region, an image of a wider region can be acquired with respect to the imaging target region. Also, image data with a lower resolution can be generated from the images of the divided regions or the images subjected to the stitching process.

Another example of the imaging method is as follows. The microscope device 5100 can first identify an imaging target region. The imaging target region may be identified so as to cover the entire region in which the biological sample exists, or may be identified so as to cover the target portion (the portion in which the target tissue section or the target cell exists) of the biological sample. Next, the microscope device 5100 scans a region (also referred to as a “divided scan region”) of the imaging target region in one direction (also referred to as a “scan direction”) in a plane perpendicular to the optical axis, and thus captures an image. After the scanning of the divided scan region is completed, the divided scan region next to the scan region is then scanned. These scanning operations are repeated until an image of the entire imaging target region is captured. As shown in FIG. 44, the microscope device 5100 identifies a region (a gray portion) in which a tissue section of the biological sample S exists, as an imaging target region Sa. The microscope device 5100 then scans a divided scan region Rs of the imaging target region Sa in the Y-axis direction. After completing the scanning of the divided scan region Rs, the microscope device 5100 then scans the divided scan region that is the next in the X-axis direction. This operation is repeated until scanning of the entire imaging target region Sa is completed. For the scanning of each divided scan region, the positional relationship between the microscope device 5100 and the sample placement unit 5104 is adjusted so that an image of the next divided scan region is captured after an image of one divided scan region is captured. The adjustment may be performed by moving the microscope device 5100, moving the sample placement unit 5104, or moving both. In this example, the imaging device that captures an image of each divided scan region may be a one-dimensional image sensor (a line sensor) or a two-dimensional image sensor (an area sensor). The signal acquisition unit 5103 may capture an image of each divided region via a magnifying optical system. Also, images of the respective divided scan regions may be continuously captured while the microscope device 5100 and/or the sample placement unit 5104 is moved. The imaging target region may be divided so that the respective divided scan regions partially overlap, or the imaging target region may be divided so that the respective divided scan regions do not overlap. A plurality of images of each divided scan region may be captured while the imaging conditions such as the focal length and/or the exposure time are changed. The information processing device can also generate image data of a wider region by stitching a plurality of adjacent divided scan regions. As the stitching process is performed on the entire imaging target region, an image of a wider region can be acquired with respect to the imaging target region. Also, image data with a lower resolution can be generated from the images of the divided scan regions or the images subjected to the stitching process.

### <6. Configuration Example of Hardware>

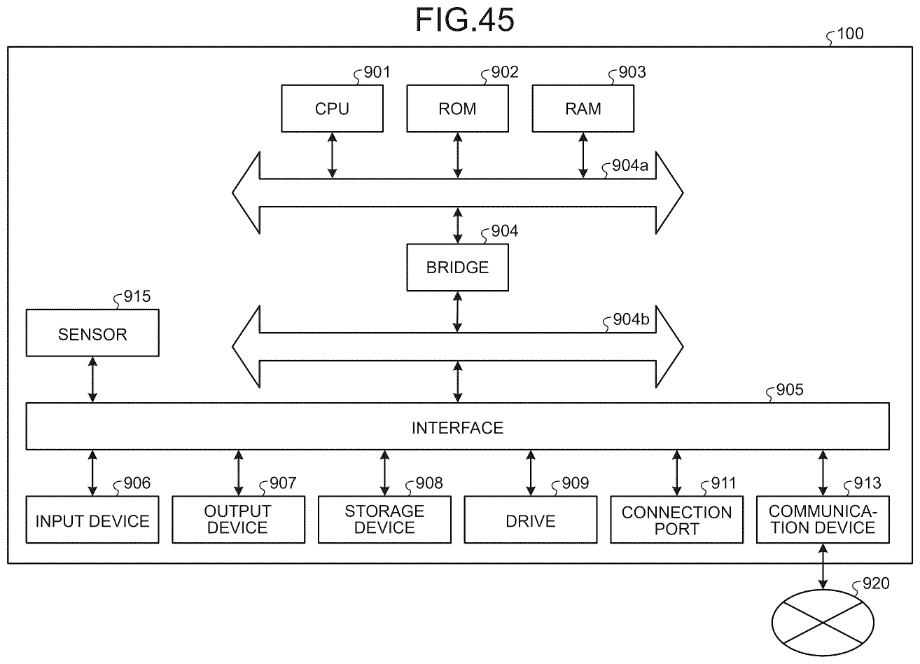

A hardware configuration example of the information processing device 100 according to each embodiment (or each modification) will be described with reference to FIG. 45. FIG. 45 is a block diagram showing an example of a schematic configuration of hardware of the information processing device 100. Various processes by the information processing device 100 are implemented, for example, by cooperation of software and hardware described below.

As shown in FIG. 45, the information processing device 100 includes a central processing unit (CPU) 901, a read only memory (ROM) 902, a random access memory (RAM) 903, and a host bus 904a. Furthermore, the information processing device 100 includes a bridge 904, an external bus 904b, an interface 905, an input device 906, an output device 907, a storage device 908, a drive 909, a connection port 911, a communication device 913, and a sensor 915. The information processing device 100 may include a processing circuit such as a DSP or an ASIC instead of or in addition to the CPU 901.

The CPU 901 functions as an arithmetic processing device and a control device, and controls the overall operation in the information processing device 100 according to various programs. In addition, the CPU 901 may be a microprocessor. The ROM 902 stores programs, operation parameters, and the like used by the CPU 901. The RAM 903 primarily stores programs used in the execution of the CPU 901, parameters that appropriately change in the execution, and the like. The CPU 901 can embody, for example, at least the processing unit 130 and the control unit 150 of the information processing device 100.

The CPU 901, the ROM 902, and the RAM 903 are mutually connected by a host bus 904a including a CPU bus and the like. The host bus 904a is connected to the external bus 904b such as a peripheral component interconnect/interface (PCI) bus via the bridge 904. Note that the host bus 904a, the bridge 904, and the external bus 904b do not necessarily need to be configured separately, and these functions may be mounted on one bus.

The input device 906 is implemented by, for example, a device to which information is input by an implementer, such as a mouse, a keyboard, a touch panel, a button, a microphone, a switch, and a lever. Furthermore, the input device 906 may be, for example, a remote control device using infrared rays or other radio waves, or may be an external connection device such as a mobile phone or a PDA corresponding to the operation of the information processing device 100. Furthermore, the input device 906 may include, for example, an input control circuit that generates an input signal on the basis of information input by the implementer using the above input units and outputs the input signal to the CPU 901. By operating the input device 906, the implementer can input various data to the information processing device and instruct the information processing device 100 to perform a processing operation. The input device 906 can embody at least the operating unit 160 of the information processing device 100, for example.

The output device 907 is formed by a device capable of visually or audibly notifying the implementer of acquired information. Examples of such a device include a display device such as a CRT display device, a liquid crystal display device, a plasma display device, an EL display device, and a lamp, a sound output device such as a speaker and a headphone, and a printer device. The output device 907 can embody at least the display unit 140 of the information processing device 100, for example.

The storage device 908 is a device for storing data. The storage device 908 is achieved by, for example, a magnetic storage device such as an HDD, a semiconductor storage device, an optical storage device, a magneto-optical storage device, or the like. The storage device 908 may include a storage medium, a recording device that records data in the storage medium, a reading device that reads data from the storage medium, a deletion device that deletes data recorded in the storage medium, and the like. The storage device 908 stores programs and various data executed by the CPU 901, various data acquired from the outside, and the like. The storage device 908 can embody at least the storage unit 120 of the information processing device 100, for example.

The drive 909 is a reader/writer for a storage medium, and is built in or externally attached to the information processing device 100. The drive 909 reads information recorded in a removable storage medium such as a mounted magnetic disk, optical disk, magneto-optical disk, or semiconductor memory, and outputs the information to the RAM 903. Furthermore, the drive 909 can also write information to a removable storage medium.

The connection port 911 is an interface connected to an external device, and is a connection port to an external device capable of transmitting data by, for example, a universal serial bus (USB).

The communication device 913 is, for example, a communication interface formed by a communication device or the like for connecting to the network 920. The communication device 913 is, for example, a communication card for wired or wireless local area network (LAN), long term evolution (LTE), Bluetooth (registered trademark), wireless USB (WUSB), or the like. Furthermore, the communication device 913 may be a router for optical communication, a router for asymmetric digital subscriber line (ADSL), a modem for various types of communication, or the like. For example, the communication device 913 can transmit and receive signals and the like to and from the Internet and other communication devices according to a predetermined protocol such as TCP/IP.

In the present embodiment, the sensor 915 includes a sensor capable of acquiring a spectrum (for example, an imaging element or the like), but may include another sensor (for example, an acceleration sensor, a gyro sensor, a geomagnetic sensor, a pressure-sensitive sensor, a sound sensor, a distance measuring sensor, or the like). The sensor 915 can embody at least the image acquisition unit 112 of the information processing device 100, for example.

Note that the network 920 is a wired or wireless transmission path of information transmitted from a device connected to the network 920. For example, the network 920 may include a public network such as the Internet, a telephone network, or a satellite communication network, various local area networks (LANs) including Ethernet (registered trademark), a wide area network (WAN), or the like. In addition, the network 920 may include a dedicated line network such as an Internet protocol-virtual private network (IP-VPN).

The hardware configuration example capable of implementing the functions of the information processing device 100 has been described above. Each of the above-described components may be implemented using a general-purpose member, or may be implemented by hardware specialized for the function of each component. Therefore, it is possible to appropriately change the hardware configuration to be used according to the technical level at the time of implementing the present disclosure.

Note that a computer program for implementing each function of the information processing device 100 as described above can be created and mounted on a PC or the like. Furthermore, it is also possible to provide a computer-readable recording medium storing such a computer program. The recording medium is, for example, a magnetic disk, an optical disk, a magneto-optical disk, a flash memory, or the like. In addition, the computer program described above may be distributed via, for example, a network without using the recording medium.

### 7. Appendix

Note that the present technology can also have the following configurations.

(1)

An information processing device comprising:
an acquisition unit that acquires a fluorescence spectrum derived from a biological sample and position information of the biological sample from a sample including the biological sample;
an identification unit that identifies, from the fluorescence spectrum, information regarding a plurality of different biomarkers of the biological sample associated with the position information of the biological sample; and
a correlation analysis unit that performs matrix decomposition processing corresponding to a combination of the plurality of biomarkers on the information regarding the plurality of biomarkers and outputs a correlation of the information regarding the plurality of biomarkers.
   (2)

The information processing device according to (1), wherein
the correlation analysis unit performs the matrix decomposition processing by joint non-negative matrix factorization (JNMF) on the information regarding the plurality of biomarkers, and then performs clustering processing.
   (3)

The information processing device according to (2), wherein
the correlation analysis unit obtains a residual sum of squares of the JNMF while changing a number of clusters in the clustering processing, and determines the number of clusters from a change tendency of the residual sum of squares.
   (4)

The information processing device according to (2), wherein
a number of clusters in the clustering processing is set by a user.
   (5)

The information processing device according to any one of (1) to (4), further comprising:
a selection unit that determines a predetermined region of the sample, wherein
the identification unit identifies information regarding the plurality of biomarkers associated with the position information of the biological sample in the predetermined region from the fluorescence spectrum of the predetermined region.
   (6)

The information processing device according to (5), wherein
the selection unit determines a plurality of the predetermined regions.
   (7)

The information processing device according to (6), wherein
the correlation analysis unit performs the matrix decomposition processing by joint non-negative matrix factorization (JNMF) on the information regarding the plurality of biomarkers, and then performs clustering processing, and
a number of clusters in the clustering processing is set according to a number of the predetermined regions.
   (8)

The information processing device according to any one of (5) to (7), wherein
the predetermined region is set by a user.
   (9)

The information processing device according to any one of (5) to (8), wherein
the selection unit determines the predetermined region at a common position of a plurality of the samples,
the acquisition unit acquires the fluorescence spectrum and the position information of the biological sample for each of the predetermined regions,
the identification unit identifies, from the fluorescence spectrum for each of the predetermined regions, information regarding the plurality of biomarkers for each of the predetermined regions associated with the position information of the biological sample for each of the predetermined regions, and
the correlation analysis unit performs the matrix decomposition processing on the information regarding the plurality of biomarkers for each of the predetermined regions, and outputs a correlation of the information regarding the plurality of biomarkers for each of the predetermined regions.
   (10)

The information processing device according to any one of (5) to (8), wherein
the selection unit determines the predetermined region of different positions of a plurality of the samples,
the acquisition unit acquires the fluorescence spectrum and the position information of the biological sample for each of the predetermined regions,
the identification unit identifies, from the fluorescence spectrum for each of the predetermined regions, information regarding the plurality of biomarkers for each of the predetermined regions associated with the position information of the biological sample for each of the predetermined regions, and
the correlation analysis unit performs the matrix decomposition processing on the information regarding the plurality of biomarkers for each of the predetermined regions, and outputs a correlation of the information regarding the plurality of biomarkers for each of the predetermined regions.
   (11)

The information processing device according to (10), wherein
the plurality of samples is a plurality of different specimens.
   (12)

The information processing device according to (11), wherein
the plurality of specimens is specimens for each of patients.
   (13)

The information processing device according to (11), wherein
the plurality of specimens is specimens for each part of a patient.
   (14)

The information processing device according to any one of (1) to (13), further comprising:
a sorting unit that changes, on a basis of an arrangement order of a plurality of pieces of unit information included in information regarding one biomarker out of the information regarding the plurality of biomarkers, an arrangement order of a plurality of pieces of unit information included in information regarding other
the correlation analysis unit performs the matrix decomposition processing on the information regarding the plurality of biomarkers in which the arrangement order has been changed, and outputs a correlation of the information regarding the plurality of biomarkers.
   (15)

The information processing device according to any one of (1) to (14), further comprising:
an information acquisition unit that acquires an administration drug candidate for a patient related to the biological sample; and
an estimation unit that estimates efficacy of the administration drug candidate to the patient from a correlation of the information regarding the plurality of biomarkers and the administration drug candidate for the patient.
   (16)

The information processing device according to (15), wherein
the estimation unit extracts a membership of a common module from the correlation of the information regarding the plurality of biomarkers, and estimates efficacy of the administration drug candidate to the patient from the membership of the common module and the administration drug candidate for the patient.
   (17)

The information processing device according to any one of (1) to (16), wherein
the information regarding the biomarkers is a degree of positive cells.
   (18)

The information processing device according to (17), wherein
the information regarding the biomarkers is a positive cell rate, a number of positive cells, or a luminance value indicating a degree of the positive cells.
   (19)

The information processing device according to (2), wherein
the correlation analysis unit gives a predetermined initial value to the JNMF.
   (20)

The information processing device according to (19), wherein
the correlation analysis unit connects a matrix X and a matrix H for each of the biomarkers, performs a least squares method on the matrix X and the matrix H for each of the biomarkers connected to acquire a matrix W, and employs the acquired matrix W as the predetermined initial value.
   (21)

The information processing device according to (19), wherein
the correlation analysis unit performs a least squares method on a matrix X and a matrix H for each of the biomarkers to acquire a matrix W for each of the biomarkers, and employs a matrix W having a smallest residual from the acquired matrix W for each of the biomarkers as the predetermined initial value.
   (22)

The information processing device according to (19), wherein
the correlation analysis unit performs a least squares method on a matrix X and a matrix H for each of the biomarkers to acquire a matrix W for each of the biomarkers, and employs an average value of the acquired matrix W for each of the biomarkers as the predetermined initial value.
   (23)

The information processing device according to (19), wherein
the predetermined initial value includes an initial value related to a new sample to be added, and
the correlation analysis unit employs a random number only for the initial value related to the new sample.
   (24)

The information processing device according to (19), wherein
the predetermined initial value includes an initial value related to a new sample to be added, and
the correlation analysis unit employs an average value of W for each of clusters of the clustering processing for the initial value related to the new sample.
   (25)

A biological sample analysis system comprising:
an imaging device that acquires a specimen image of a sample including a biological sample; and
an information processing device that processes the specimen image, wherein
the information processing device includes
an acquisition unit that acquires a fluorescence spectrum derived from the biological sample and position information of the biological sample from the specimen image,
an identification unit that identifies, from the fluorescence spectrum, information regarding a plurality of different biomarkers of the biological sample associated with the position information of the biological sample, and
a correlation analysis unit that performs matrix decomposition processing corresponding to a combination of the plurality of biomarkers on the information regarding the plurality of biomarkers and outputs a correlation of the information regarding the plurality of biomarkers.
   (26)

A biological sample analysis method comprising:
acquiring a fluorescence spectrum derived from a biological sample and position information of the biological sample from a sample including the biological sample;
identifying, from the fluorescence spectrum, information regarding a plurality of different biomarkers of the biological sample associated with the position information of the biological sample; and
performing matrix decomposition processing corresponding to a combination of the plurality of biomarkers on the information regarding the plurality of biomarkers and outputting a correlation of the information regarding the plurality of biomarkers.
   (27)

A biological sample analysis system including the information processing device according to any one of (1) to (24).

(28)

A biological sample analysis method for performing analysis by the information processing device according to any one of (1) to (24).

### REFERENCE SIGNS LIST

1 OBSERVATION UNIT
2 PROCESSING UNIT
3 DISPLAY UNIT
10 EXCITATION UNIT
10A FLUORESCENT REAGENT
11A REAGENT IDENTIFICATION INFORMATION
20 STAGE
20A SPECIMEN
21 STORAGE UNIT
21A SPECIMEN IDENTIFICATION INFORMATION
22 DATA CALIBRATION UNIT
23 IMAGE FORMATION UNIT
30 SPECTRAL IMAGING UNIT
30A FLUORESCENCE STAINED SPECIMEN
40 OBSERVATION OPTICAL SYSTEM
50 SCANNING MECHANISM
60 FOCUS MECHANISM
70 NON-FLUORESCENCE OBSERVING UNIT
80 CONTROL UNIT
100 INFORMATION PROCESSING DEVICE
110 ACQUISITION UNIT
111 INFORMATION ACQUISITION UNIT
112 IMAGE ACQUISITION UNIT
120 STORAGE UNIT
121 INFORMATION STORAGE UNIT
122 IMAGE INFORMATION STORAGE UNIT
123 ANALYSIS RESULT STORAGE UNIT
130 PROCESSING UNIT
131 ANALYSIS UNIT
132 IMAGE GENERATION UNIT
133 SPACE ANALYSIS UNIT
133a SELECTION UNIT
133b IDENTIFICATION UNIT
133c SORTING UNIT
133d CORRELATION ANALYSIS UNIT
133e ESTIMATION UNIT
140 DISPLAY UNIT
150 CONTROL UNIT
160 OPERATING UNIT
200 DATABASE
500 FLUORESCENCE OBSERVATION DEVICE
5000 MICROSCOPE SYSTEM
5100 MICROSCOPE DEVICE
5101 LIGHT IRRADIATION UNIT
5102 OPTICAL UNIT
5103 SIGNAL ACQUISITION UNIT
5104 SAMPLE PLACEMENT UNIT
5110 CONTROL UNIT
5120 INFORMATION PROCESSING UNIT

1. An information processing device comprising:
an acquisition unit that acquires a fluorescence spectrum derived from a biological sample and position information of the biological sample from a sample including the biological sample;
an identification unit that identifies, from the fluorescence spectrum, information regarding a plurality of different biomarkers of the biological sample associated with the position information of the biological sample; and
a correlation analysis unit that performs matrix decomposition processing corresponding to a combination of the plurality of biomarkers on the information regarding the plurality of biomarkers and outputs a correlation of the information regarding the plurality of biomarkers.

2. The information processing device according to claim 1, wherein
the correlation analysis unit performs the matrix decomposition processing by joint non-negative matrix factorization (JNMF) on the information regarding the plurality of biomarkers, and then performs clustering processing.

3. The information processing device according to claim 2, wherein
the correlation analysis unit obtains a residual sum of squares of the JNMF while changing a number of clusters in the clustering processing, and determines the number of clusters from a change tendency of the residual sum of squares.

4. The information processing device according to claim 2, wherein
a number of clusters in the clustering processing is set by a user.

5. The information processing device according to claim 1, further comprising:
a selection unit that determines a predetermined region of the sample, wherein
the identification unit identifies information regarding the plurality of biomarkers associated with the position information of the biological sample in the predetermined region from the fluorescence spectrum of the predetermined region.

6. The information processing device according to claim 5, wherein
the selection unit determines a plurality of the predetermined regions.

7. The information processing device according to claim 6, wherein
the correlation analysis unit performs the matrix decomposition processing by joint non-negative matrix factorization (JNMF) on the information regarding the plurality of biomarkers, and then performs clustering processing, and
a number of clusters in the clustering processing is set according to a number of the predetermined regions.

8. The information processing device according to claim 5, wherein
the predetermined region is set by a user.

9. The information processing device according to claim 5, wherein
the selection unit determines the predetermined region at a common position of a plurality of the samples,
the acquisition unit acquires the fluorescence spectrum and the position information of the biological sample for each of the predetermined regions,
the identification unit identifies, from the fluorescence spectrum for each of the predetermined regions, information regarding the plurality of biomarkers for each of the predetermined regions associated with the position information of the biological sample for each of the predetermined regions, and
the correlation analysis unit performs the matrix decomposition processing on the information regarding the plurality of biomarkers for each of the predetermined regions, and outputs a correlation of the information regarding the plurality of biomarkers for each of the predetermined regions.

10. The information processing device according to claim 5, wherein
the selection unit determines the predetermined region of different positions of a plurality of the samples,
the acquisition unit acquires the fluorescence spectrum and the position information of the biological sample for each of the predetermined regions,
the identification unit identifies, from the fluorescence spectrum for each of the predetermined regions, information regarding the plurality of biomarkers for each of the predetermined regions associated with the position information of the biological sample for each of the predetermined regions, and
the correlation analysis unit performs the matrix decomposition processing on the information regarding the plurality of biomarkers for each of the predetermined regions, and outputs a correlation of the information regarding the plurality of biomarkers for each of the predetermined regions.

11. The information processing device according to claim 10, wherein
the plurality of samples is a plurality of different specimens.

12. The information processing device according to claim 11, wherein
the plurality of specimens is specimens for each of patients.

13. The information processing device according to claim 11, wherein
the plurality of specimens is specimens for each part of a patient.

14. The information processing device according to claim 1, further comprising:
a sorting unit that changes, on a basis of an arrangement order of a plurality of pieces of unit information included in information regarding one biomarker out of the information regarding the plurality of biomarkers, an arrangement order of a plurality of pieces of unit information included in information regarding other biomarkers, wherein
the correlation analysis unit performs the matrix decomposition processing on the information regarding the plurality of biomarkers in which the arrangement order has been changed, and outputs a correlation of the information regarding the plurality of biomarkers.

15. The information processing device according to claim 1, further comprising:
an information acquisition unit that acquires an administration drug candidate for a patient related to the biological sample; and
an estimation unit that estimates efficacy of the administration drug candidate to the patient from a correlation of the information regarding the plurality of biomarkers and the administration drug candidate for the patient.

16. The information processing device according to claim 15, wherein
the estimation unit extracts a membership of a common module from the correlation of the information regarding the plurality of biomarkers, and estimates efficacy of the administration drug candidate to the patient from the membership of the common module and the administration drug candidate for the patient.

17. The information processing device according to claim 1, wherein
the information regarding the biomarkers is a degree of positive cells.

18. The information processing device according to claim 17, wherein
the information regarding the biomarkers is a positive cell rate, a number of positive cells, or a luminance value indicating a degree of the positive cells.

19. The information processing device according to claim 2, wherein
the correlation analysis unit gives a predetermined initial value to the JNMF.

20. The information processing device according to claim 19, wherein
the correlation analysis unit connects a matrix X and a matrix H for each of the biomarkers, performs a least squares method on the matrix X and the matrix H for each of the biomarkers connected to acquire a matrix W, and employs the acquired matrix W as the predetermined initial value.

21. The information processing device according to claim 19, wherein
the correlation analysis unit performs a least squares method on a matrix X and a matrix H for each of the biomarkers to acquire a matrix W for each of the biomarkers, and employs a matrix W having a smallest residual from the acquired matrix W for each of the biomarkers as the predetermined initial value.

22. The information processing device according to claim 19, wherein
the correlation analysis unit performs a least squares method on a matrix X and a matrix H for each of the biomarkers to acquire a matrix W for each of the biomarkers, and employs an average value of the acquired matrix W for each of the biomarkers as the predetermined initial value.

23. The information processing device according to claim 19, wherein
the predetermined initial value includes an initial value related to a new sample to be added, and
the correlation analysis unit employs a random number only for the initial value related to the new sample.

24. The information processing device according to claim 19, wherein
the predetermined initial value includes an initial value related to a new sample to be added, and
the correlation analysis unit employs an average value of W for each of clusters of the clustering processing for the initial value related to the new sample.

25. A biological sample analysis system comprising:
an imaging device that acquires a specimen image of a sample including a biological sample; and
an information processing device that processes the specimen image, wherein
the information processing device includes
an acquisition unit that acquires a fluorescence spectrum derived from the biological sample and position information of the biological sample from the specimen image,
an identification unit that identifies, from the fluorescence spectrum, information regarding a plurality of different biomarkers of the biological sample associated with the position information of the biological sample, and
a correlation analysis unit that performs matrix decomposition processing corresponding to a combination of the plurality of biomarkers on the information regarding the plurality of biomarkers and outputs a correlation of the information regarding the plurality of biomarkers.

26. A biological sample analysis method comprising:
acquiring a fluorescence spectrum derived from a biological sample and position information of the biological sample from a sample including the biological sample;
identifying, from the fluorescence spectrum, information regarding a plurality of different biomarkers of the biological sample associated with the position information of the biological sample; and
performing matrix decomposition processing corresponding to a combination of the plurality of biomarkers on the information regarding the plurality of biomarkers and outputting a correlation of the information regarding the plurality of biomarkers.
